SEQUENCE LISTING (9 sequences)

SEQ ID NO: 1 - protein, 16 aa, synthetic construct
  VARIANT 4: note = Any naturally or non-naturally occurring amino acid
  VARIANT 12: note = Any naturally or non-naturally occurring amino acid
  VARIANT 14: note = Any naturally or non-naturally occurring amino acid
QRFXTGHFGGLXPXNG

SEQ ID NO: 2 - protein, 16 aa, synthetic construct
QRFCTGHFGGLYPCNG

SEQ ID NO: 3 - protein, 16 aa, synthetic construct
QRFCTGHFGGLHPCNG

SEQ ID NO: 4 - protein, 16 aa, synthetic construct
QRFVTGHFGGLYPANG

SEQ ID NO: 5 - protein, 16 aa, synthetic construct
QRFVTGHFGGLHPANG

SEQ ID NO: 6 - protein, 15 aa, synthetic construct
GGGGSGGGGSGGGGS

SEQ ID NO: 7 - protein, 5 aa, synthetic construct
GGGGS

SEQ ID NO: 8 - protein, 14 aa, synthetic construct
YVPKEFNAETFTFH

SEQ ID NO: 9 - protein, 6 aa, synthetic construct
HHHHHH

(19) United States
(12) Patent Application Publication
(10) Pub. No.: US 2024/0076381 A1
(43) Pub. Date: Mar. 7, 2024

(54) FCRN BINDING POLYPEPTIDES

(71) Applicant: THE BOARD OF TRUSTEES OF THE UNIVERSITY OF ILLINOIS, URBANA, IL (US)

(72) Inventors: SHANNON J. SIRK, URBANA, IL (US); VINCE W. KELLY, URBANA, IL (US)

(73) Assignee: THE BOARD OF TRUSTEES OF THE UNIVERSITY OF ILLINOIS, URBANA, IL (US)

(21) Appl. No.: 18/453,098

(22) Filed: Aug. 21, 2023

Related U.S. Application Data
(60) Provisional application No. 63/400,107, filed on Aug. 23, 2022.

(51) Int. Cl. C07K 16/28; A61K 35/74

(52) U.S. Cl. CPC C07K 16/283; A61K 35/74; A61K 2035/11; C07K 2317/622

(57) ABSTRACT

The invention relates to FcRn polypeptides, to antibodies and related fragments thereof containing the FcRn polypeptides, to production of said antibodies and fragments and to use of said antibodies and fragments for therapy of various conditions.

### PRIORITY

This application claims the benefit of U.S. Ser. No. 63/400,107, filed Aug. 23, 2022, which is incorporated by reference herein in its entirety.

### SEQUENCE LISTING

The instant application contains a Sequence Listing which has been submitted electronically in XML format and is hereby incorporated by reference in its entirety. Said XML copy, created on Aug. 25, 2023, is named 745145_UIUC-040_SL.xml and is 9,885 bytes in size.

### BACKGROUND

Therapeutic antibodies have become one of the most widely used classes of biotherapeutics due to their unique antigen specificity and their ability to be engineered against diverse disease targets. There is significant interest in utilizing truncated or portions/fragments of antibodies as therapeutics, as their small size affords favorable properties such as increased tumor penetration as well as the ability to utilize lower-cost prokaryotic production methods. Their small size and simple architecture, however, also lead to rapid blood clearance, limiting the efficacy of these potentially powerful therapeutics. There is a need for compositions and methods for overcoming these limitations imposed by the small size of such antibody therapeutics.

### SUMMARY

An aspect provides an isolated polypeptide comprising a neonatal Fc receptor (FcRn) binding polypeptide comprising an amino acid sequence set forth in QRFX₁TGHFGGLX₂PX₃NG (SEQ ID NO:1) or YVPKEFNAETFTFH (SEQ ID NO:8) and a specific binding moiety. The specific binding moiety can comprise a V_{H} domain, a V_{H} domain and a V_{L} domain, an antibody fragment, a single domain antibody, a V_{HH}, or a nanobody. The FcRn binding polypeptide can comprise an amino acid sequence as set forth in SEQ ID NO:2, 3, 4, or 5.

Provided herein are polypeptides that include, from an N-terminus to a C-terminus (a) an antibody light chain variable region (V_{L}) domain, a first neonatal Fc receptor (FcRn) binding polypeptide comprising an amino acid sequence set forth in QRFX₁TGHFGGLX₂PX₃NG (SEQ ID NO:1), and an antibody heavy chain variable region (V_{H}) domain; or (b) an antibody light chain variable region (V_{L}) domain, a first FcRn binding polypeptide comprising an amino acid sequence set forth in QRFX₁TGHFGGLX₂PX₃NG (SEQ ID NO:1), an antibody heavy chain variable region (V_{H}) domain; and a second binding FcRn binding polypeptide comprising an amino acid sequence set forth in QRFX₁TGHFGGLX₂PX₃NG (SEQ ID NO:1); or (c) an antibody light chain variable region (V_{L}) domain, a linker, an antibody heavy chain variable region (V_{H}) domain; and a first FcRn binding polypeptide comprising an amino acid sequence set forth in QRFX₁TGHFGGLX₂PX₃NG (SEQ ID NO:1).

In some embodiments, the first FcRn binding polypeptide is an amino acid sequence as set forth in SEQ ID NO:2, 3, 4, or 5. In some embodiments, the second FcRn binding polypeptide is an amino acid sequence as set forth in SEQ ID NO:2, 3, 4, or 5.

The polypeptide can include a first FcRn binding polypeptide with an amino acid sequence as set forth in SEQ ID NO:2 and the second FcRn binding polypeptide with an amino acid sequence as set forth in SEQ ID NO:2. In some embodiments, the polypeptide can include a first FcRn binding polypeptide with an amino acid sequence as set forth in SEQ ID NO:2 and a second FcRn binding polypeptide with an amino acid sequence as set forth in SEQ ID NO:3. The polypeptide can include a first FcRn binding polypeptide with an amino acid sequence as set forth in SEQ ID NO:2 and a second FcRn binding polypeptide with an amino acid sequence as set forth in SEQ ID NO:4. In some embodiments, the polypeptide can include a first FcRn binding polypeptide with an amino acid sequence as set forth in SEQ ID NO:2 and a second FcRn binding polypeptide with an amino acid sequence as set forth in SEQ ID NO:5. In some embodiments, the polypeptide can include a first FcRn binding polypeptide with an amino acid sequence as set forth in SEQ ID NO:3 and a second FcRn binding polypeptide with an amino acid sequence as set forth in SEQ ID NO:2. In some embodiments, the polypeptide can include a first FcRn binding polypeptide with an amino acid sequence as set forth in SEQ ID NO:3 and a second FcRn binding polypeptide with an amino acid sequence as set forth in SEQ ID NO:3. In some embodiments, the polypeptide can include a first FcRn binding polypeptide with an amino acid sequence as set forth in SEQ ID NO:3 and a second FcRn binding polypeptide with an amino acid sequence as set forth in SEQ ID NO:4. In some embodiments, the polypeptide can include a first FcRn binding polypeptide with an amino acid sequence as set forth in SEQ ID NO:3 and a second FcRn binding polypeptide with an amino acid sequence as set forth in SEQ ID NO:5. In some embodiments, the polypeptide can include a first FcRn binding polypeptide with an amino acid sequence as set forth in SEQ ID NO:4 and a second FcRn binding polypeptide with an amino acid sequence as set forth in SEQ ID NO:2. In some embodiments, the polypeptide can include a first FcRn binding polypeptide with an amino acid sequence as set forth in SEQ ID NO:4 and a second FcRn binding polypeptide with an amino acid sequence as set forth in SEQ ID NO:3. In some embodiments, the polypeptide can include a first FcRn binding polypeptide with an amino acid sequence as set forth in SEQ ID NO:4 and a second FcRn binding polypeptide with an amino acid sequence as set forth in SEQ ID NO:4. In some embodiments, the polypeptide can include a first FcRn binding polypeptide with an amino acid sequence as set forth in SEQ ID NO:4 and a second FcRn binding polypeptide with an amino acid sequence as set forth in SEQ ID NO:5. In some embodiments, the polypeptide can include a first FcRn binding polypeptide with an amino acid sequence as set forth in SEQ ID NO:5 and the second FcRn binding polypeptide with an amino acid sequence as set forth in SEQ ID NO:2. In some embodiments, the polypeptide can include a first FcRn binding polypeptide with an amino acid sequence as set forth in SEQ ID NO:5 and a second FcRn binding polypeptide with an amino acid sequence as set forth in SEQ ID NO:3. In some embodiments, the polypeptide can include a first FcRn binding polypeptide with an amino acid sequence as set forth in SEQ ID NO:5 and a second FcRn binding polypeptide with an amino acid sequence as set forth in SEQ ID NO:4. In some embodiments, the polypeptide can include a first FcRn binding polypeptide with an amino acid sequence as set forth in SEQ ID NO:5 and a second FcRn binding polypeptide comprises an amino acid sequence as set forth in SEQ ID NO:5.

The antibody light chain variable region (V_{L}) domain or the antibody heavy chain variable region (V_{H}) domain, or both can be human or humanized.

Also provided herein is a single chain fragment variable (scFv) antibody comprising an amino acid sequence set forth in SEQ ID NO:1 (QRFX₁TGHFGGLX₂PX₃NG). The present disclosure also provides vectors vector comprising a polynucleotide encoding SEQ ID NO:1 (QRFX₁TGHFGGLX₂PX₃NG) or the polypeptides described herein. Also provided are polynucleotides encoding the polypeptides or the vectors described herein.

Provided herein are methods of treatment using a therapeutic antibody that includes an amino acid sequence set forth in SEQ ID NO:1 (QRFX₁TGHFGGLX₂PX₃NG) to a subject in need thereof. The therapeutic antibody can include a V_{L} domain and a V_{H} domain from 3F8, Abagovomab, Abciximab, Abituzumab, Abrezekimab, Abrilumab, Actoxumab, Adalimumab, Adecatumumab, Aducanumab, Afasevikumab, Afelimomab, Alacizumab pegol, Alemtuzumab, Alirocumab, Altumomab pentetate, Amatuximab, Am ivantamab, Anatumomab mafenatox, Andecaliximab, Anetumab ravtansine, Anifrolumab, Ansuvimab, Anrukinzumab, Apolizumab, Aprutumab ixadotin, Arcitumomab, Ascrinvacumab, Aselizumab, Atezolizumab, Atidortoxumab, Atinumab, Atoltivimab, Atoltivimab/maftivimab/odesivimab, Atorolimumab, Avelumab, Azintuxizumab vedotin, Bamlanivimab, Bapineuzumab, Basiliximab, Bavituximab, Bebtelovimab, Bectumomab, Begelomab, Belantamab mafodotin, Belimumab, Bemarituzumab, Benralizumab, Berlimatoxumab, Bermekimab, Bersanlimab, Bertilimumab, Besilesomab, Bevacizumab, Bezlotoxumab, Biciromab, Bimagrumab, Bimekizumab, Birtamimab, Bivatuzumab, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Cam idanlumab tesirine, Camrelizumab, Canakinumab, Cantuzumab mertansine, Cantuzumab ravtansine, Caplacizumab, Casirivimab, Capromab, Carlumab, Carotuximab, Catumaxomab, Cedelizumab, Cemiplimab, Cergutuzumab amunaleukin, Certolizumab pegol, Cetrelimab, Cetuximab, Cibisatamab, Cilgavimab, Cirmtuzumab, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, Crizanlizumab, Crotedumab, Cusatuzumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dezamizumab, Dinutuximab, Dinutuximab beta, Diridavumab, Domagrozumab, Dorlimomab aritox, Dostarlimab, Drozitumab, Duligotuzumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emapalumab, Emibetuzumab, Emicizumab, Enapotamab vedotin, Enavatuzumab, Enfortumab vedotin, Enlimomab pegol, Enoblituzumab, Enokizumab, Enoticumab, Ensituximab, Epcoritamab, Epitumomab cituxetan, Epratuzumab, Eptinezumab, Erenumab, Erlizumab, Ertumaxomab, Etaracizumab, Etesevimab, Etigilimab, Etrolizumab, Evinacumab, Evolocumab, Exbivirumab, Fanolesomab, Faralimomab, Faricimab, Farletuzumab, Fasinumab, Felvizumab, Fezakinumab, Fibatuzumab, Ficlatuzumab, Figitumumab, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, Gancotamab, Ganitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, lanalumab, Ibalizumab, Sintilimab, Ibritumomab tiuxetan, Icrucumab, Idarucizumab, Ifabotuzumab, Igovomab, Iladatuzumab vedotin, Imalumab, Imaprelimab, Imciromab, Imdevimab, Imgatuzumab, Inclacumab, Indatuximab ravtansine, Indusatumab vedotin, Inebilizumab, Infliximab, Intetumumab, Inolimomab, Inotuzumab ozogamicin, Ipilimumab, Iomab-B, Iratumumab, Isatuximab, Iscalimab, Istiratumab, Itolizumab, Ixekizumab, Keliximab, Labetuzumab, Lacnotuzumab, Ladiratuzumab vedotin, Lampalizumab, Lanadelumab, Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lupartumab, Lupartumab amadotin, Lutikizumab, Maftivimab, Mapatumumab, Margetuximab, Marstacimab, Maslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD, Nacolomab tafenatox, Nam ilumab, Naptumomab estafenatox, Naratuximab emtansine, Narnatumab, Natalizumab, Navicixizumab, Navivumab, Naxitamab, Nebacumab, Necitumumab, Nemolizumab, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odesivimab, Odulimomab, Ofatumumab, Olaratumab, Oleclumab, Olendalizumab, Olokizumab, Omalizumab, Omburtamab, Onartuzumab, Ontuxizumab, Onvatilimab, Opicinumab, Oportuzumab monatox, Oregovomab, Orticumab, Otelixizumab, Otilimab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Prezalumab, Plozalizumab, Pogalizumab, Polatuzumab vedotin, Ponezumab, Porgaviximab, Prasinezumab, Prezalizumab, Priliximab, Pritoxaximab, Pritumumab, Quilizumab, Racotumomab, Radretumab, Rafivirumab, Ralpancizumab, Ramucirumab, Ranevetmab, Ranibizumab, Raxibacumab, Ravagalimab, Ravulizumab, Refanezumab, Regavirumab, Regdanvimab, Relatlimab, Remtolumab, Reslizumab, Rilotumumab, Rinucumab, Risankizumab, Rituximab, Rivabazumab pegol, Robatumumab, Rmab, Roledumab, Rom ilkimab, Romosozumab, Rontalizumab, Rosmantuzumab, Rovalpituzumab tesirine, Rovelizumab, Rozanolixizumab, Ruplizumab, Sacituzumab govitecan, Samalizumab, Samrotamab vedotin, Sarilumab, Satralizumab, Satumomab pendetide, Secukinumab, Selicrelumab, Seribantumab, Setoxaximab, Setrusumab, Sevirumab, Sibrotuzumab, Sifalimumab, Siltuximab, Simtuzumab, Siplizumab, Sirtratumab vedotin, Sirukumab, Sofituzumab vedotin, Solanezumab, Solitomab, Sonepcizumab, Sontuzumab, Sotrovimab, Spartalizumab, Stamulumab, Sulesomab, Suptavumab, Sutimlimab, Suvizumab, Suvratoxumab, Tabalumab, Tacatuzumab tetraxetan, Tadocizumab, Tafasitamab, Talacotuzumab, Talizumab, Talquetamab, Tamtuvetmab, Tanezumab, Taplitumomab paptox, Tarextumab, Tavolimab, Teclistamab, Tefibazumab, Telimomab aritox, Telisotuzumab, Telisotuzumab vedotin, Tenatumomab, Teneliximab, Teplizumab, Tepoditamab, Teprotumumab, Tesidolumab, Tetulomab, Tezepelumab, Tibulizumab, Tildrakizumab, Tigatuzumab, Tim igutuzumab, Timolumab, Tiragolumab, Tiragotumab, Tislelizumab, Tisotumab vedotin, Tixagevimab, Tocilizumab, Tomuzotuximab, Toralizumab, Tosatoxumab, Tositumomab, Tovetumab, Tralokinumab, Trastuzumab, Trastuzumab duocarmazine, Trastuzumab emtansine, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Vunakizumab, Xentuzumab, Zalutumumab, Zanolimumab, Zatuximab, Zenocutuzumab, Ziralimumab, Zolbetuximab, or Zolimomab aritox. The methods of treatment can also involve delivering the pharmaceutical compositions described herein to a subject in need thereof.

Also provided herein are polypeptides comprising an amino acid sequence set forth in SEQ ID NO:1 (QRFX₁TGHFGGLX₂PX₃NG), wherein the polypeptide is not naturally occurring. In some embodiments, X₁ is C or V. In some embodiments, X₂ is Y or H. In some embodiments, X₃ is C or A. As a non-limiting example, the polypeptide is QRFVTGHFGGLHPANG (SEQ ID NO:5).

### BRIEF DESCRIPTION OF THE DRAWINGS

Various objectives, features, and advantages of the disclosed subject matter can be more fully appreciated with reference to the following detailed description of the disclosed subject matter when considered in connection with the following drawings, in which like reference numerals identify like elements.

The patent or application file contains at least one drawing executed in color. Copies of this patent or patent application publication with color drawing(s) will be provided by the Office upon request and payment of the necessary fee.

FIG. 1 shows schematic representation of FcRn binding polypeptide modified scFvs described herein. V_{L} indicates variable light domain; V_{H} indicates variable heavy domain; L represents linker (also shown as unlabeled boxes flanking linker peptides); Cyc indicates cyclic peptide; Lin indicates linear peptide; and Alb indicates albumin peptide.

FIG. 2 shows representative ELISA data from five of the FcRn polypeptide-modified scFvs used in this study showing FcRn binding at pH 6 and 7.4, with unmodified scFv and full-length IgG controls. Assays were performed in triplicate. Error bars represent one standard deviation.

FIG. 3A shows recycling in T84 cell-based assays relative to full-length IgG control. Stars indicate significance over the unmodified scFv negative transport control. FIG. 3B shows recycling in the presence or absence of 12 μM (30-fold excess) IgG or human serum albumin. Data for scFvs modified with FcRn binding polypeptides is presented as ng recycled, relative to IgG control without added competitor. Data for scFv modified with FcRn binding polypeptides-Alb is presented as ng recycled relative to full-length albumin control without added competitor. FIG. 3C-FIG. 3E show recycling relative to full-length IgG control of scFvs modified with Cyc or CycY12H FcRn polypeptides-Fc as an intra-linker or C-terminal extension (FIG. 3C); Lin or LinY12H FcRn binding polypeptides-Fc as an intra-linker or C-terminal extension (FIG. 3D), or two FcRn binding polypeptides-Fc (FIG. 3E). All experiments (FIG. 3A-3E) were performed in triplicate. * 0.01 p 0.05; ** 0.001 p 0.01; *** 0.0001 p 0.001; **** p<0.0001 by two-sided student's t-test in GraphPad Prism 9.

FIG. 4A shows trans-membrane transcytosis of scFvs modified with FcRn binding polypeptides or FcRn polypeptides-Alb, relative to full-length IgG control. Stars indicate significance over the unmodified scFv negative transport control. FIG. 4B shows transcytosis of scFv modified with FcRn polypeptides-Albumin, relative to full-length human serum albumin. Stars indicate significance over the unmodified scFv negative transport control. FIG. 4C shows transcytosis of scFvs modified with Y12H mutant or non-mutant FcRn polypeptides, relative to full-length IgG control. All experiments in FIG. 4A-4C were performed in triplicate. * 0.01≤p≤0.05; ** 0.001≤p≤0.01; *** 0.0001≤p≤0.001; **** p<0.0001 by two-sided student's t-test in GraphPad Prism 9.

FIG. 5 panels A-D show In vitro characterization of FcRnBP-modified sdAbs. A, B. Binding of 1 μM aTcdA (A) and αTNF (B) to FcRn at pH 6 and 7.4. C, D. FcRn-mediated recycling activity of aTcdA (C) and αTNF (D) relative to the unmodified control antibody fragments. Data shown is the mean of two separate experiments. All samples were run in triplicate. * 0.01 p 0.05; ** 0.001 p 0.01; *** 0.0001 p 0.001; **** p<0.0001 by two-sided student's t-test in GraphPad Prism 9.

FIG. 6 shows A20.1 sdAb fused to albumin-mimicking peptide (SEQ ID NO:8) (AlbP-V_{HH}) or A20.1 with no modification (V_{HH}) delivered interperitoneally or by rectal infusion to transgenic mice.

The drawings are not necessarily to scale, or inclusive of all elements of a system, emphasis instead generally being placed upon illustrating the concepts, structures, and techniques sought to be protected herein.

### DETAILED DESCRIPTION

The details of one or more variations of the subject matter described herein are set forth in the accompanying drawings and the description below. Other features and advantages of the subject matter described herein will be apparent from the description and drawings, and from the claims. The disclosed subject matter is not, however, limited to any particular embodiment disclosed.

Overview

Over the past several decades, antibody-based therapeutics have been developed to treat a number of diverse diseases. With an ever-expanding market and steady pace of product approvals, the therapeutic and economic impact of this class of drugs continues to grow as researchers further engineer these complex biomolecules to improve performance and decrease cost. Many of these efforts have advanced to clinical use, including the generation of antibody-drug conjugates to enhance potency; fusion of select antibody domains to improve the pharmacokinetics of active biologicals; and the development of engineered antibody fragments for imaging and therapy. Due to their size, small antibody fragments in particular offer a number of advantages over larger, full-length antibodies, including improved tissue penetration and more efficient and cost effective production in prokaryotic systems.

Despite these advantages, therapeutic development of small antibody fragments remains particularly challenging compared to full-length antibodies because, in order to reduce size, most fragments lack an Fc (crystallizable fragment) domain. In addition to mediating immune effector function such as recruiting humoral and cellular responses, the Fc domain is also responsible for extending the serum half-life of antibodies via engagement with the neonatal Fc receptor (FcRn), promoting recycling rather than degradation of these long-lived serum proteins. FcRn also binds albumin in a similar way (but at an orthogonal binding site). When IgG or albumin are taken up by polarized epithelial or endothelial cells, they are packaged into endosomes, which acidify as they traffic through the cell. In this low-pH environment, histidines on albumin and the IgG Fc domain are protonated and binding to FcRn and its cognate light chain μ2 microglobulin (μ2m) is enabled. Association of μ2m with FcRn is required for binding with IgG and albumin. These albumin- or IgG-FcRn-μ2m complexes are then trafficked away from the lysosomal fate and are either recycled back to the membrane from which they entered or transcytosed to the opposite membrane, where they are released upon encountering the physiological pH of the extracellular environment. Small antibody fragments lacking an Fc domain are unable to engage FcRn and are thus rapidly cleared from the circulation over several hours rather than persisting for several weeks as with full length antibodies or native albumin.

A common approach to circumvent these limitations is to enable engagement with the half-life extending neonatal Fc receptor (FcRn). This is usually achieved via fusion with a large Fc domain, which negates the benefits of the antibody fragment's small size.

To solve these problems, the present disclosure provides FcRn binding polypeptides that mimic the FcRn binding domains of IgG or albumin without dramatically increasing the size of an antibody fragment or portion. These FcRn binding polypeptides resemble native IgG engagement with FcRn and FcRn-mediated recycling and trans-membrane transcytosis in cell-based assays. Also provided herein are polypeptides from human serum albumin that enable FcRn-mediated function when grafted onto antibodies, single-chain variable fragment (scFv), single variable domain antibodies and their fragments (V_{HH}, sdAb, nanobody) scaffolds. Thus, the present disclosure provides approaches for the selection of polypeptides from full-length proteins that could enable the transfer of non-native functions to small recombinant proteins without significantly impacting their size or structure.

Polypeptides

Provided herein are polypeptides that are useful in the present disclosure. “Polypeptide” as used herein refers to any peptide or protein comprising two or more amino acids joined to each other by peptide bonds or modified peptide bonds, i.e., peptide isosteres. Polypeptides refers to both short chains, commonly referred to as peptides, oligopeptides or oligomers, and to longer chains, generally referred to as proteins. Polypeptides can contain amino acids other than the 20 gene-encoded amino acids. Polypeptides can include amino acid sequences modified either by natural processes, such as posttranslational processing, or by chemical modification techniques that are well known in the art.

Polypeptides described herein can be derived from other proteins or polypeptide (herein referred to as a starting polypeptide). They can have one or more mutations relative to the starting polypeptide, e.g., one or more amino acid residues which have been substituted with another amino acid residue or which has one or more amino acid residue insertions or deletions. The polypeptide can comprise an amino acid sequence which is not naturally occurring. Such variations necessarily have less than 100% sequence identity or similarity with the starting polypeptide. In one embodiment, the variant will have an amino acid sequence from about 60% to less than 100% amino acid sequence identity or similarity with the amino acid sequence of the starting polypeptide. In another embodiment, the variant will have an amino acid sequence from about 75% to less than 100%), from about 80% to less than 100%, from about 85% to less than 100%, from about 90% to less than 100%), from about 95% to less than 100% amino acid sequence identity or similarity with the amino acid sequence of the starting polypeptide.

Modifications can occur anywhere in a polypeptide, including the peptide backbone, the amino acid side-chains and the amino or carboxyl termini. The same type of modification can be present in the same or to varying degrees at several sites in a given polypeptide. The polypeptide can include one or more types of modifications. Polypeptides can be branched as a result of ubiquitination, and they can be cyclic, with or without branching. Cyclic, branched and branched cyclic polypeptides can result from post translation natural processes or can be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

FcRn Binding Polypeptides

Polypeptides of the disclosure can be FcRn binding polypeptides. As used herein, the term “FcRn binding polypeptide” refers to a polypeptide which can, in part or whole, bind to, interact with or otherwise mimic the engagement of a protein (e.g., IgG or albumin) with FcRn. In some embodiments, the FcRn binding polypeptide can include a portion of an Fc region that mediates binding to FcRn. FcRn binding polypeptides can also be derived from albumin. In some embodiments a FcRn binding polypeptide is a synthetic Fc mimic that binds to the same or similar epitope of FcRn as does the IgG Fc domain. A synthetic Fc mimic can be derived from, for example, a phage display library.

In some embodiments, the FcRn binding polypeptides are not naturally occurring.

In an aspect, an isolated polypeptide comprising a neonatal Fc receptor (FcRn) binding polypeptide comprising an amino acid sequence set forth in QRFX₁TGHFGGLX₂PX₃NG (SEQ ID NO:1) or YVPKEFNAETFTFH (SEQ ID NO:8) and a specific binding moiety are provided. A specific binding moiety can comprise, for example, a V_{H} domain, a V_{H} domain and a V_{L} domain, an antibody fragment, or a nanobody. An FcRn binding polypeptide can comprise an amino acid sequence as set forth in SEQ ID NO:2, 3, 4, or 5.

In some embodiments, FcRn binding polypeptides described herein comprise an amino acid sequence set forth in SEQ ID NO:1 (QRFX₁TGHFGGLX₂PX₃NG). Optionally, the polypeptide is not naturally occurring. X1, X2, X3, can be any naturally or non-naturally occurring amino acid. For example, X₁ can be the amino acid denoted by the single letter code C or V. X2 can be the amino acid denoted by the single letter code Y or H. X3 can be the amino acid denoted by the single letter code C or A. Exemplary FcRn binding polypeptides include the amino acid sequence as set forth in QRFCTGHFGGLYPCNG (SEQ ID NO:2); QRFCTGHFGGLHPCNG (SEQ ID NO:3); QRFVTGHFGGLYPANG (SEQ ID NO:4); QRFVTGHFGGLHPANG (SEQ ID NO:5). FcRn binding polypeptides can include one or more additional amino acids at the N terminus or C terminus of SEQ ID NO:2, 3, 4 or 5. One or more amino acids can be removed from the N terminus or the C terminus of SEQ ID NO:2, 3, 4, or 5. FcRn binding polypeptides of the disclosure can have from about 60% identity to 100% identity to the sequence of SEQ ID NO:2, 3, 4 or 5, for example from about 60%-70%, 70%-80%, 80%-90%, 90%-100% identity to SEQ ID NO:2, 3, 4, or 5.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100).

In some embodiments the length of a comparison sequence aligned for comparison purposes is at least 80% of the length of the reference sequence (e.g. SEQ ID NO:1), and in some embodiments is at least 85%, 90%, 91%, 92%, 93%, 95%, 96%, 97%, 98%, 99% or 100% of the length of the reference sequence. In an embodiment, the two sequences are the same length. Therefore, wherein a length of a comparison sequence is required to be at least 100% of the length of a reference sequence, and a reference sequence is 10 amino acids in length and a comparison sequence is 100 amino acids in length, and wherein the 10 amino acids of the reference sequence contiguously align with 10 of the 100 amino acids of the comparison sequence, the sequence identity of the comparison sequence is only 10%.

The small size of the polypeptides described herein are important for favorable properties such as increased tumor penetration as well as the ability to utilize lower-cost prokaryotic production methods. In an aspect, a polypeptide (e.g., SEQ ID NO:1-5 and 8) is smaller than about 50, 40, 30, 20, 19, 18, 17, 16, 15, or 14 amino acids in length.

Ranges of desired degrees of sequence identity are approximately 80% to 100% and integer values in between. Percent identities between a disclosed sequence and a claimed sequence can be at least 80%, at least 83%, at least 85%, at least 90%, at least 95%, at least 98%, at least 99%, at least 99.5%, or at least 99.9%. In general, an exact match indicates 100% identity over the length of the reference sequence (e.g., SEQ ID NO:1 or 8).

Polypeptides and polynucleotides that are sufficiently similar to polypeptides and polynucleotides described herein can be used herein. Polypeptides and polynucleotides that are about 90, 91, 92, 93, 94 95, 96, 97, 98, 99 99.5% or more identical to polypeptides and polynucleotides described herein can also be used herein.

For example, a polynucleotide can have 80% 85%, 90%, 91%, 92%, 93%, 94%, 95%, 96%, 97%, 98% 99% or more identity to SEQ ID NO:1-5, or 8.

Polypeptides of the disclosure can include one, two, three or more FcRn polypeptides. When more than one FcRn binding polypeptide is present it can be the same FcRn polypeptide, or it can be a different FcRn polypeptide. For example, when the polypeptides of the disclosure include two FcRn polypeptides, their identity can be as follows: (a) the first FcRn binding polypeptide comprises an amino acid sequence as set forth in SEQ ID NO:2 and the second FcRn binding polypeptide comprises an amino acid sequence as set forth in SEQ ID NO:2; (b) the first FcRn binding polypeptide comprises an amino acid sequence as set forth in SEQ ID NO:2 and the second FcRn binding polypeptide comprises an amino acid sequence as set forth in SEQ ID NO:3; (c) the first FcRn binding polypeptide comprises an amino acid sequence as set forth in SEQ ID NO:2 and the second FcRn binding polypeptide comprises an amino acid sequence as set forth in SEQ ID NO:4; (d) the first FcRn binding polypeptide comprises an amino acid sequence as set forth in SEQ ID NO:2 and the second FcRn binding polypeptide comprises an amino acid sequence as set forth in SEQ ID NO:5; (e) the first FcRn binding polypeptide comprises an amino acid sequence as set forth in SEQ ID NO:3 and the second FcRn binding polypeptide comprises an amino acid sequence as set forth in SEQ ID NO:2; (f) the first FcRn binding polypeptide comprises an amino acid sequence as set forth in SEQ ID NO:3 and the second FcRn binding polypeptide comprises an amino acid sequence as set forth in SEQ ID NO:3; (g) the first FcRn binding polypeptide comprises an amino acid sequence as set forth in SEQ ID NO:3 and the second FcRn binding polypeptide comprises an amino acid sequence as set forth in SEQ ID NO:4; (h) the first FcRn binding polypeptide comprises an amino acid sequence as set forth in SEQ ID NO:3 and the second FcRn binding polypeptide comprises an amino acid sequence as set forth in SEQ ID NO:5; (i) the first FcRn binding polypeptide comprises an amino acid sequence as set forth in SEQ ID NO:4 and the second FcRn binding polypeptide comprises an amino acid sequence as set forth in SEQ ID NO:2; (j) the first FcRn binding polypeptide comprises an amino acid sequence as set forth in SEQ ID NO:4 and the second FcRn binding polypeptide comprises an amino acid sequence as set forth in SEQ ID NO:3; (k) the first FcRn binding polypeptide comprises an amino acid sequence as set forth in SEQ ID NO:4 and the second FcRn binding polypeptide comprises an amino acid sequence as set forth in SEQ ID NO:4; (I) the first FcRn binding polypeptide comprises an amino acid sequence as set forth in SEQ ID NO:4 and the second FcRn binding polypeptide comprises an amino acid sequence as set forth in SEQ ID NO:5; (m) the first FcRn binding polypeptide comprises an amino acid sequence as set forth in SEQ ID NO:5 and the second FcRn binding polypeptide comprises an amino acid sequence as set forth in SEQ ID NO:2; (n) the first FcRn binding polypeptide comprises an amino acid sequence as set forth in SEQ ID NO:5 and the second FcRn binding polypeptide comprises an amino acid sequence as set forth in SEQ ID NO:3; (o) the first FcRn binding polypeptide comprises an amino acid sequence as set forth in SEQ ID NO:5 and the second FcRn binding polypeptide comprises an amino acid sequence as set forth in SEQ ID NO:4; and/or (p) the first FcRn binding polypeptide comprises an amino acid sequence as set forth in SEQ ID NO:5 and the second FcRn binding polypeptide comprises an amino acid sequence as set forth in SEQ ID NO:5.

FcRn binding polypeptide can be derived from albumin. In some embodiments, the FcRn binding polypeptide can be a region or portion of albumin. In an embodiment, an FcRn binding polypeptide derived from albumin can be YVPKEFNAETFTFH (SEQ ID NO:8)

Antibodies and Specific Binding Moieties

Polypeptides described herein can include antibodies, specific binding fragments of antibodies, and immunoglobulins. Antibodies, specific binding fragments of antibodies, immunoglobulins, and other antibody-like molecules described below can be considered “specific binding moieties.” As used herein the terms “antibodies” (Abs) and “immunoglobulins” (Igs) refer to glycoproteins having the same structural characteristics. While antibodies exhibit binding specificity to a specific antigen, immunoglobulins include both antibodies and other antibody-like molecules. “Antibody,” as used herein, encompasses any polypeptide comprising an antigen-binding site regardless of the source, species of origin, method of production, and characteristics. Antibodies include immunoglobulin molecules and immunologically active portions of immunoglobulin molecules, i.e., molecules that contain an antigen binding site that immunospecifically binds an antigen. Antibodies and specific binding moieties include natural or artificial, mono- or polyvalent antibodies including, but not limited to, polyclonal, monoclonal, multispecific, human, humanized or chimeric antibodies, single-chain antibodies, single-chain Fvs (scFv), disulfide-linked Fvs (sdfv), tandem di-scFvs, tandem tri-scFvs, minibodies, nanobodies, diabodies, tribodies, tetrabodies, immunoglobulin single variable domains (ISV), such as V_{HH} (including humanized V_{HH}), a camelized V_{H}, a single domain antibody, a domain antibody, or a dAb, and antibody fragments. “Antibody fragments” can be “specific binding moieties” and include a portion of an intact antibody, such as the antigen binding or variable region of the intact antibody. Examples of antibody fragments include, e.g., Fab, Fab′ and F(ab′)2, Fc fragments or Fc-fusion products, fragments including either a V_{L} or V_{H} domain, and Fv fragments. and the like (Zapata et al. Protein Eng. 8(10):1057-1062 [1995]).

“Native antibodies” and “intact immunoglobulins,” or the like, are usually heterotetrameric glycoproteins of about 150,000 daltons, composed of two identical light (L) chains and two identical heavy (H) chains. The light chains from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA, and IgA2. The heavy-chain constant domains that correspond to the different classes of immunoglobulins are called α, δ, ε, γ, and μ, respectively. The subunit structures and three-dimensional configurations of different classes of immunoglobulins are well known. Polypeptides and constructs described herein can be derived from IgA, IgD, IgE, IgG, and IgM, and any immunoglobulin subclass(isotypes), e.g., IgG1, IgG2, IgG3, IgG4, IgA, and IgA2. Polypeptides and constructs described herein can be derived from any heavy-chain constant domains that correspond to the different classes of immunoglobulins, e.g., α, δ, ε, γ, and μ,

Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains. Each variable region includes three segments called complementarity-determining regions (CDRs) or hypervariable regions and a more highly conserved portions of variable domains are called the framework region (FR). The variable domains of heavy and light chains each includes four FR regions, largely adopting a p-sheet configuration, connected by three CDRs, which form loops connecting, and in some cases forming part of the p-sheet structure. The CDRs in each chain are held together in close proximity by the FRs and, with the CDRs from the other chain, contribute to the formation of the antigen-binding site of antibodies (see Kabat et al., NIH Publ. No. 91-3242, Vol. I, pages 647-669 [1991]). The constant domains are not involved directly in binding an antibody to an antigen, but exhibit various effector functions, such as participation of the antibody in antibody dependent cellular cytotoxicity.

Experimentally, antibodies can be cleaved with the proteolytic enzyme papain, which causes each of the heavy chains to break, producing three separate antibody fragments. The two units that comprise a light chain and a fragment of the heavy chain are approximately equal in mass to the light chain and are called the Fab fragments (i.e., the “antigen binding” fragments). The third unit, consisting of two equal segments of the heavy chain, is called the Fc fragment. The Fc fragment is typically not involved in antigen-antibody binding but is important in later processes involved in ridding the body of the antigen.

“Fv” is the minimum antibody fragment which contains a complete antigen-recognition and antigen-binding site. This region consists of a dimer of one heavy-chain variable domain and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although in some cases at a lower affinity than the entire binding site. Single variable domain antibodies and their fragments (V_{HH}, sdAb, nanobody, etc.) can also bind antigen with equal affinity as, e.g., V_{H}-V_{L} dimers having only 3 CDRs.

In some embodiments, polypeptides of the disclosure can include scFvs. “Single-chain Fv” or “sFv” or “scFv” antibody fragments comprise the V_{H} and V_{L} domains of antibody, wherein these domains are present in a single polypeptide chain. An Fv polypeptide can further comprise a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds., Springer-Verlag, New York, pp. 269-315 (1994).

Specific binding moieties, antibodies, specific binding fragments thereof, and immunoglobulins as disclosed herein can “specifically bind” to an antigen, which means that the specific binding moieties, antibodies, specific binding fragments thereof, and immunoglobulins as disclosed herein can form a complex with an antigen that is relatively stable under physiologic conditions. Specific binding can be characterized by an equilibrium dissociation constant of at least about 1×10⁻⁷ M or less (e.g., a smaller Ko denotes a tighter binding). Methods for determining whether two molecules specifically bind include, for example, equilibrium dialysis, surface plasmon resonance, and the like.

An isolated polypeptide can comprise a neonatal Fc receptor (FcRn) binding polypeptide comprising an amino acid sequence set forth in QRFX₁TGHFGGLX₂PX₃NG (SEQ ID NO:1) and a specific binding moiety. The specific binding moiety can comprise, e.g., a V_{H} domain, a V_{H} domain and a V_{L} domain, an antibody fragment, a nanobody, or any other antibody or immunoglobulin molecule described herein.

The isolated polypeptide of claim 1, wherein the FcRn binding polypeptide comprises an amino acid sequence as set forth in SEQ ID NO:2, 3, 4, or 5.

Polypeptides described herein can include from an N-terminus to a C-terminus, a V_{L} domain, a first FcRn binding polypeptide comprising an amino acid sequence set forth in QRFX₁TGHFGGLX₂PX₃NG (SEQ ID NO:1), and an antibody heavy chain. Polypeptides of the disclosure can include an antibody light chain variable region (V_{L}) domain, a first FcRn binding polypeptide comprising an amino acid sequence set forth in QRFX₁TGHFGGLX₂PX₃NG (SEQ ID NO:1), an antibody heavy chain variable region (V_{H}) domain; and a second FcRn binding polypeptide comprising an amino acid sequence set forth in QRFX₁TGHFGGLX₂PX₃NG (SEQ ID NO:1). In some embodiments, polypeptides of the disclosure can include an antibody light chain variable region (V_{L}) domain, a linker, an antibody heavy chain variable region (V_{H}) domain; and a first FcRn binding polypeptide comprising an amino acid sequence set forth in QRFX₁TGHFGGLX₂PX₃NG (SEQ ID NO:1). The scFvs described herein can include SEQ ID NO:1.

Polypeptides described herein can include one or more linkers. A linker refers to a moiety that links or connects together one or more portions or regions of a polypeptide or one or more polypeptides. The linker can be a peptide linker that includes from about 1-100 amino acids (e.g., about 1, 5, 10, 15, 20, 30, 40, 50, 60, 70, 80, 90, 100 or more amino acids). The type of linker can vary depending on the crystal structure of the antibody heavy chain and antibody light chain. The linkers can be derived from naturally occurring sequences of amino acids. Alternatively, the linkers can be artificially designed peptide linker. In some embodiments, the linker can be composed of flexible residues such as, Glycine (G) and Serine (S), so that the adjacent polypeptide domains are free to move relative to one another. Non limiting examples of linkers include (Gly₄Ser)₃ linkers (SEQ ID NO: 6), e.g., GGGGSGGGGSGGGGS. (SEQ ID NO:6) or a single repeat of GGGGS linker (SEQ ID NO:7).

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 [1985]). However, these fragments can now be produced directly by recombinant host cells. For example, the antibody fragments can be isolated from the antibody phage libraries. Alternatively, Fab′-SH fragments can be directly recovered from E. coli and chemically coupled to form F(ab′)2 fragments (Carter et al., Bio/Technology 10:163-167 [1992]). According to another approach, F(ab′)2 fragments can be isolated directly from recombinant host cell culture. Other techniques for the production of antibody fragments will be apparent to the skilled practitioner. In other embodiments, an antibody is a single chain Fv fragment (scFv). See WO 93/16185.

Polypeptides of the disclosure can include monoclonal antibodies. The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the antibodies, fragments thereof, and polypeptides described herein can be made by the hybridoma method first described by Kohler et al., Nature, 256:495 (1975), or can be made by recombinant DNA methods (see, e.g., U.S. Pat. No. 4,816,567). The antibodies, fragments thereof, and polypeptides as described herein can also be isolated from phage antibody libraries using the techniques described in Clackson et al., Nature, 352:624-628 (1991) and Marks et al., J. Mol. Biol., 222:581-597 (1991), for example.

Antibodies (e.g., scFvs) described herein can be humanized. “Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and maximize antibody performance. In general, a humanized antibody or specific binding fragment thereof can comprise substantially all of at least one, and typically two, variable domains, in which all or substantially all of the CDRs correspond to those of a non-human immunoglobulin and all or substantially all of the FRs are those of a human immunoglobulin sequence. A humanized antibody or specific binding fragment thereof can also comprise at least a portion of an immunoglobulin constant region (Fc), typically that of a human immunoglobulin. For further details, see Jones et al., Nature, 321:522-525 (1986); Reichmann et al., Nature, 332:323-329 (1988); and Presta, Curr. Op. Struct. Biol., 2:593-596 (1992). A humanized antibody includes a PRIMATIZED™ antibody wherein the antigen-binding region of the antibody is derived from an antibody produced by immunizing macaque monkeys with the antigen of interest.

Antibodies and specific binding fragments thereof described herein can be human antibodies. A human antibody and specific binding fragments thereof, as used herein, refers to an antibody or specific binding fragment that comprises human immunoglobulin protein sequences only. A human antibody and specific binding fragments thereof can contain murine carbohydrate chains if produced in a mouse, in a mouse cell, or in a hybridoma derived from a mouse cell. A human antibody and specific binding fragments thereof can contain rat carbohydrate chains if produced in a rat, in a rat cell, or in a hybridoma derived from a rat cell. In some embodiments, a human antibody and specific binding fragments thereof can be isolated from the serum of a human. In some embodiments, a human antibody and specific binding fragments thereof can be artificially prepared. In some embodiments, a human antibody and specific binding fragments thereof can be a human monoclonal antibody. The process of generating human antibodies can start with, for example, phage display technology or animal immunizations. When mice are utilized, they are injected with the designated therapeutic target (e.g., protein), specific antibodies to the target are identified, and cells, such as Chinese hamster ovary (CHO) cells, are used to produce the monoclonal antibodies (mAbs). Human mAbs can also be developed in transgenic mice that have been genetically engineered with the human immunoglobulin locus.

Non-limiting examples of antibodies include, Abrilumab, Actoxumab, Adalimumab, Adecatumumab, Aducanumab, Afasevikumab, Alirocumab, Am ivantamab, Anetumab ravtansine, Anifrolumab, Ansuvimab, Aprutumab ixadotin, Ascrinvacumab, Atidortoxumab, Atinumab, Atoltivimab, Atoltivimab/maftivimab/odesivimab, Atorolimumab, Avelumab, Bamlanivimab, BCD-, Bebtelovimab, Belimumab, Berlimatoxumab, Bermekimab, Bersanlimab, Bertilimumab, Bezlotoxumab, Bimagrumab, Bleselumab, Brazikumab, Briakinumab, Brodalumab, Burosumab, Camidanlumab tesirine, Canakinumab, Casirivimab, Carlumab, Cemiplimab, Cetrelimab, Cilgavimab, Cixutumumab, Conatumumab, Crotedumab, CR, Daratumumab, Dectrekumab, Denosumab, Diridavumab, Drozitumab, Dupilumab, Durvalumab, Dusigitumab, Efungumab, Eldelumab, Elezanumab, Elgemtumab, Emapalumab, Enapotamab vedotin, Enfortumab vedotin, Enoticumab, Epcoritamab, Erenumab, Etesevimab, Evinacumab, Evolocumab, Exbivirumab, Fasinumab, Fezakinumab, Figitumumab, Firivumab, Flanvotumab, Fletikumab, Foralumab, Foravirumab, Fresolimumab, Fulranumab, Gancotamab, Ganitumab, Gantenerumab, Gedivumab, Gimsilumab, Glembatumumab vedotin, Golimumab, Guselkumab, lanalumab, Sintilimab, Icrucumab, Imalumab, Imdevimab, Inclacumab, Indusatumab vedotin, Intetumumab, Ipilimumab, Iratumumab, Iscalimab, Istiratumab, Lanadelumab, Lenzilumab, Lerdelimumab, Lesofavumab, Lexatumumab, Libivirumab, Lirilumab, Lucatumumab, Lupartumab, Lupartumab amadotin, Maftivimab, Mapatumumab, Marstacimab, Mavrilimumab, Metelimumab, Morolimumab, Namilumab, Narnatumab, Navivumab, Nebacumab, Necitumumab, Nesvacumab, Nirsevimab, Nivolumab, Odesivimab, Ofatumumab, Olaratumab, Oleclumab, OMS, Onvatilimab, Opicinumab, Orticumab, Otilimab, Oxelumab, Pamrevlumab, Panitumumab, Panobacumab, Patritumab, Placulumab, Prezalumab, Pritumumab, Radretumab, Rafivirumab, Ramucirumab, Raxibacumab, Regavirumab, Regdanvimab, Relatlimab, Remtolumab, Rilotumumab, Rinucumab, Robatumumab, Rmab, Roledumab, Sarilumab, Secukinumab, Selicrelumab, Seribantumab, Setrusumab, Sevirumab, SHP, Sifalimumab, Sirtratumab vedotin, Sirukumab, Sotrovimab, Stamulumab, Suptavumab, Suvratoxumab, Tabalumab, Talquetamab, Tarextumab, Teclistamab, Tepoditamab, Teprotumumab, Tesidolumab, Tezepelumab, Timolumab, Tiragolumab, Tiragotumab, Tisotumab vedotin, Tixagevimab, Tosatoxumab, Tovetumab, Tralokinumab, Tremelimumab, Trevogrumab, Tuvirumab, Ulocuplumab, Urelumab, Ustekinumab, Utomilumab, Vantictumab, Varisacumab, Varlilumab, Vesencumab, Votumumab, Zalutumumab, Zanolimumab, or Ziralimumab.

Antibodies and specific binding fragments thereof can be made, for example, via traditional hybridoma techniques, recombinant DNA methods, or phage display techniques using antibody libraries. For various other antibody production techniques, see Antibodies: A Laboratory Manual, eds. Harlow et al., Cold Spring Harbor Laboratory, 1988.

In some embodiments, polypeptides described herein can include therapeutic antibodies or V_{L} and V_{H} regions of the same. Any antibody or antibody fragment that is useful in the treatment of one or more diseases or disorders can be considered a therapeutic antibody. Therapeutic antibodies are widely used in the treatment of cancer, autoimmunity, and inflammatory diseases or for drug delivery to target antigen, most of which are monoclonal antibodies. Therapeutic antibodies recognize and bind to an antigen receptor to activate or inhibit a series of biological process, e.g., for blocking cancer cell growth or triggering immune system. V_{L} and V_{H} regions of any antibody, including therapeutic antibodies can be used in the constructs of this disclosure, including, for example the V_{L} and V_{H} regions of 3F8, Abagovomab, Abciximab, Abituzumab, Abrezekimab, Abrilumab, Actoxumab, Adalimumab, Adecatumumab, Aducanumab, Afasevikumab, Afelimomab, Alacizumab pegol, Alemtuzumab, Alirocumab, Altumomab pentetate, Amatuximab, Am ivantamab, Anatumomab mafenatox, Andecaliximab, Anetumab ravtansine, Anifrolumab, Ansuvimab, Anrukinzumab, Apolizumab, Aprutumab ixadotin, Arcitumomab, Ascrinvacumab, Aselizumab, Atezolizumab, Atidortoxumab, Atinumab, Atoltivimab, Atoltivimab/maftivimab/odesivimab, Atorolimumab, Avelumab, Azintuxizumab vedotin, Bamlanivimab, Bapineuzumab, Basiliximab, Bavituximab, Bebtelovimab, Bectumomab, Begelomab, Belantamab mafodotin, Belimumab, Bemarituzumab, Benralizumab, Berlimatoxumab, Bermekimab, Bersanlimab, Bertilimumab, Besilesomab, Bevacizumab, Bezlotoxumab, Biciromab, Bimagrumab, Bimekizumab, Birtamimab, Bivatuzumab, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Cam idanlumab tesirine, Camrelizumab, Canakinumab, Cantuzumab mertansine, Cantuzumab ravtansine, Caplacizumab, Casirivimab, Capromab, Carlumab, Carotuximab, Catumaxomab, Cedelizumab, Cemiplimab, Cergutuzumab amunaleukin, Certolizumab pegol, Cetrelimab, Cetuximab, Cibisatamab, Cilgavimab, Cirmtuzumab, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, Crizanlizumab, Crotedumab, Cusatuzumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dezamizumab, Dinutuximab, Dinutuximab beta, Diridavumab, Domagrozumab, Dorlimomab aritox, Dostarlimab, Drozitumab, Duligotuzumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emapalumab, Emibetuzumab, Emicizumab, Enapotamab vedotin, Enavatuzumab, Enfortumab vedotin, Enlimomab pegol, Enoblituzumab, Enokizumab, Enoticumab, Ensituximab, Epcoritamab, Epitumomab cituxetan, Epratuzumab, Eptinezumab, Erenumab, Erlizumab, Ertumaxomab, Etaracizumab, Etesevimab, Etigilimab, Etrolizumab, Evinacumab, Evolocumab, Exbivirumab, Fanolesomab, Faralimomab, Faricimab, Farletuzumab, Fasinumab, Felvizumab, Fezakinumab, Fibatuzumab, Ficlatuzumab, Figitumumab, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, Gancotamab, Ganitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, lanalumab, Ibalizumab, Sintilimab, Ibritumomab tiuxetan, Icrucumab, Idarucizumab, Ifabotuzumab, Igovomab, Iladatuzumab vedotin, Imalumab, Imaprelimab, Imciromab, Imdevimab, Imgatuzumab, Inclacumab, Indatuximab ravtansine, Indusatumab vedotin, Inebilizumab, Infliximab, Intetumumab, Inolimomab, Inotuzumab ozogamicin, Ipilimumab, Iomab-B, Iratumumab, Isatuximab, Iscalimab, Istiratumab, Itolizumab, Ixekizumab, Keliximab, Labetuzumab, Lacnotuzumab, Ladiratuzumab vedotin, Lampalizumab, Lanadelumab, Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lupartumab, Lupartumab amadotin, Lutikizumab, Maftivimab, Mapatumumab, Margetuximab, Marstacimab, Maslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD, Nacolomab tafenatox, Nam ilumab, Naptumomab estafenatox, Naratuximab emtansine, Narnatumab, Natalizumab, Navicixizumab, Navivumab, Naxitamab, Nebacumab, Necitumumab, Nemolizumab, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odesivimab, Odulimomab, Ofatumumab, Olaratumab, Oleclumab, Olendalizumab, Olokizumab, Omalizumab, Omburtamab, Onartuzumab, Ontuxizumab, Onvatilimab, Opicinumab, Oportuzumab monatox, Oregovomab, Orticumab, Otelixizumab, Otilimab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Prezalumab, Plozalizumab, Pogalizumab, Polatuzumab vedotin, Ponezumab, Porgaviximab, Prasinezumab, Prezalizumab, Priliximab, Pritoxaximab, Pritumumab, Quilizumab, Racotumomab, Radretumab, Rafivirumab, Ralpancizumab, Ramucirumab, Ranevetmab, Ranibizumab, Raxibacumab, Ravagalimab, Ravulizumab, Refanezumab, Regavirumab, Regdanvimab, Relatlimab, Remtolumab, Reslizumab, Rilotumumab, Rinucumab, Risankizumab, Rituximab, Rivabazumab pegol, Robatumumab, Rmab, Roledumab, Rom ilkimab, Romosozumab, Rontalizumab, Rosmantuzumab, Rovalpituzumab tesirine, Rovelizumab, Rozanolixizumab, Ruplizumab, Sacituzumab govitecan, Samalizumab, Samrotamab vedotin, Sarilumab, Satralizumab, Satumomab pendetide, Secukinumab, Selicrelumab, Seribantumab, Setoxaximab, Setrusumab, Sevirumab, Sibrotuzumab, Sifalimumab, Siltuximab, Simtuzumab, Siplizumab, Sirtratumab vedotin, Sirukumab, Sofituzumab vedotin, Solanezumab, Solitomab, Sonepcizumab, Sontuzumab, Sotrovimab, Spartalizumab, Stamulumab, Sulesomab, Suptavumab, Sutimlimab, Suvizumab, Suvratoxumab, Tabalumab, Tacatuzumab tetraxetan, Tadocizumab, Tafasitamab, Talacotuzumab, Talizumab, Talquetamab, Tamtuvetmab, Tanezumab, Taplitumomab paptox, Tarextumab, Tavolimab, Teclistamab, Tefibazumab, Telimomab aritox, Telisotuzumab, Telisotuzumab vedotin, Tenatumomab, Teneliximab, Teplizumab, Tepoditamab, Teprotumumab, Tesidolumab, Tetulomab, Tezepelumab, Tibulizumab, Tildrakizumab, Tigatuzumab, Tim igutuzumab, Timolumab, Tiragolumab, Tiragotumab, Tislelizumab, Tisotumab vedotin, Tixagevimab, Tocilizumab, Tomuzotuximab, Toralizumab, Tosatoxumab, Tositumomab, Tovetumab, Tralokinumab, Trastuzumab, Trastuzumab duocarmazine, Trastuzumab emtansine, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Vunakizumab, Xentuzumab, Zalutumumab, Zanolimumab, Zatuximab, Zenocutuzumab, Ziralimumab, Zolbetuximab, and/or Zolimomab aritox.

Polynucleotides

The present disclosure provides polynucleotides comprising a nucleic acid sequence that encodes any of the polypeptides as described herein, and host cells into which the nucleic acids that are used are introduced to replicate the polypeptide-encoding nucleic acids and/or to express the polypeptides. In some embodiments, the host cell is eukaryotic, for example, a human cell.

Polynucleotides described herein can encode the FcRn polypeptides. For example, the polynucleotides can encode one or more polypeptides of SEQ ID NO:1-5 and 8. Polynucleotides described herein can also encode one or more of (a) an antibody light chain variable region (V_{L}) domain, a first neonatal Fc receptor (FcRn) binding polypeptide comprising an amino acid sequence set forth in QRFX₁TGHFGGLX₂PX₃NG (SEQ ID NO:1), and an antibody heavy chain variable region (V_{H}) domain; (b) an antibody light chain variable region (V_{L}) domain, a first FcRn binding polypeptide comprising an amino acid sequence set forth in QRFX₁TGHFGGLX₂PX₃NG (SEQ ID NO:1), an antibody heavy chain variable region (V_{H}) domain; and a second FcRn binding polypeptide comprising an amino acid sequence set forth in QRFX₁TGHFGGLX₂PX₃NG (SEQ ID NO:1); or (c) an antibody light chain variable region (V_{L}) domain, a linker, an antibody heavy chain variable region (V_{H}) domain; and a first FcRn binding polypeptide comprising an amino acid sequence set forth in QRFX₁TGHFGGLX₂PX₃NG (SEQ ID NO:1).

Polynucleotides can be single-stranded or double-stranded. In some embodiments, the polynucleotide is DNA. In particular embodiments, the polynucleotide is cDNA. In some embodiments, the polynucleotide is RNA. In some embodiments, the polynucleotide is included within a nucleic acid construct. In some modalities, the construct is a replicable vector. In some embodiments, the vector is selected from a plasmid, a viral vector, a phagemid, a yeast chromosomal vector and a non-episomal mammal vector.

In some embodiments, a polynucleotide is operationally linked to one or more regulatory nucleotide sequences in an expression construct.

Unless otherwise indicated, the term polynucleotide, nucleic acid molecule, or gene includes reference to the specified sequence as well as the complementary sequence thereof. Polynucleotides can be present as a single-stranded or double-stranded and linear or covalently circularly closed molecule. As used herein, a polynucleotide can include both naturally occurring and non-naturally occurring nucleotides.

Polynucleotides can be obtained from nucleic acid molecules present in, for example, a mammalian cell. Polynucleotides can also be synthesized in the laboratory, for example, using an automatic synthesizer. Polynucleotides can be isolated. An isolated polynucleotide can be a naturally occurring polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid molecules naturally found immediately flanking the recombinant DNA molecule in a naturally occurring genome is removed or absent. Isolated polynucleotides also include non-naturally occurring nucleic acid molecules. “Isolated polynucleotides” can be (i) amplified in vitro, for example via polymerase chain reaction (PCR), (ii) produced recombinantly by cloning, (iii) purified, for example, by cleavage and separation by gel electrophoresis, (iv) synthesized, for example, by chemical synthesis, or (vi) extracted from a sample.

Polynucleotides can encode full-length polypeptides, polypeptide fragments, and variant or fusion polypeptides. Polynucleotides can comprise coding sequences for naturally occurring polypeptides or can encode altered sequences that do not occur in nature. Polynucleotides can be purified free of other components, such as proteins, lipids and other polynucleotides. For example, the polynucleotide can be 50%, 75%, 90%, 95%, 96%, 97%, 98%, 99%, or 100% purified. A polynucleotide existing among hundreds to millions of other polynucleotide molecules within, for example, cDNA or genomic libraries, or gel slices containing a genomic DNA restriction digest are not to be considered a purified polynucleotide.

Vectors

The polypeptides described herein, e.g., starting polypeptides and modified polypeptides can be produced by recombinant methods. For example, a polynucleotide sequence encoding a polypeptide can be inserted into a suitable expression vector for recombinant expression. Where a polypeptide is an antibody, polynucleotides encoding additional light and heavy chain variable regions, optionally linked to constant regions, can be inserted into the same or different expression vector. An affinity tag sequence (e.g., a His(6) tag (SEQ ID NO: 9)) can optionally be attached or included within the starting polypeptide sequence to facilitate downstream purification. The DNA segments encoding immunoglobulin chains are the operably linked to control sequences in the expression vector(s) that ensure the expression of polypeptides. Expression control sequences include, but are not limited to, promoters (e.g., naturally-associated or heterologous promoters), signal sequences, enhancer elements, and transcription termination sequences. The expression control sequences can be eukaryotic promoter systems in vectors capable of transforming or transfecting eukaryotic host cells. Alternatively, the expression control sequences can be prokaryotic promoter systems. Once the vector has been incorporated into the appropriate host, the host is maintained under conditions suitable for high level expression of the nucleotide sequences, and the collection and purification of the polypeptide.

Expression vectors are typically replicable in the host organisms either as episomes or as an integral part of the host chromosomal DNA. Commonly, expression vectors contain selection markers (e.g., ampicillin-resistance, hygromycin-resistance, tetracycline resistance or neomycin resistance) to permit detection of those cells transformed with the desired DNA sequences.

Escherichia coli can be useful for cloning the polynucleotides (e.g., DNA sequences). Other microbial hosts suitable for use include bacilli, such as Bacillus subtilus, and other enterobacteriaceae, such as Salmonella, Serratia, and various Pseudomonas species. Other microbes, such as yeast, are also useful for expression. Saccharomyces and Pichia are exemplary yeast hosts, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for methanol, maltose, and galactose utilization.

In addition to microorganisms, mammalian tissue culture can also be used to express and produce the polypeptides described herein (e.g., polynucleotides encoding immunoglobulins or fragments thereof).

Expression vectors for mammalian cells can be used. Non-limiting examples of mammalian cells include, CHO cell lines, various Cos cell lines, HeLa cells, 293 cells, myeloma cell lines, and/or transformed B-cells. Expression vectors can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Expression control sequences can be promoters derived from immunoglobulin genes, SV40, adenovirus, bovine papilloma virus, cytomegalovirus and the like.

The vectors containing polynucleotide sequences of interest (e.g., SEQ ID NOs:1-5 and 8, heavy and light chain encoding sequences, and expression control sequences) can be transferred into the host cell by well-known methods, which vary depending on the type of cellular host. For example, calcium chloride transfection is commonly utilized for prokaryotic cells, whereas calcium phosphate treatment, electroporation, lipofection, biolistics or viral-based transfection can be used for other cellular hosts. (See generally Sambrook et al., Molecular Cloning: A Laboratory Manual (Cold Spring Harbor Press, 2nd ed., 1989). Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

Polypeptides can be expressed using a single vector or two vectors. In one embodiment, signal sequences can be used to facilitate expression of polypeptides described herein.

Pharmaceutical Compositions

A therapeutic or pharmaceutical composition can include at least one of the polypeptides described herein in a pharmaceutically acceptable carrier. A “pharmaceutically acceptable carrier” refers to at least one component of a pharmaceutical preparation that is normally used for administration of active ingredients. As such, a carrier can contain any pharmaceutical excipient used in the art and any form of vehicle for administration. The compositions can be, for example, injectable solutions, aqueous suspensions or solutions, non-aqueous suspensions or solutions, solid and liquid oral formulations, salves, gels, ointments, intradermal patches, creams, lotions, tablets, capsules, sustained release formulations, and the like. Additional excipients can include, for example, colorants, taste-masking agents, solubility aids, suspension agents, compressing agents, enteric coatings, sustained release aids, and the like.

The form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation can also include other carriers, adjuvants, or nontoxic, nontherapeutic, nonimmunogenic stabilizers and the like.

Pharmaceutical compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249:1527, 1990 and Hanes, Advanced Drug Delivery Reviews 28:97, 1997).

Pharmaceutical compositions of the disclosure can also include a population of recombinant bacteria comprising the polypeptides or polynucleotides encoding the polypeptides of the disclosure and/or the vectors described herein. The recombinant bacteria can have environmentally limited viability and/or activity. The recombinant bacteria can have sensitivity to one or more antibiotic agents. In certain embodiments, the viability and/or activity of the recombinant bacteria in a human host is dependent on a compound not found naturally in healthy humans. In certain embodiments, the viability and/or activity of the recombinant bacteria in a human host is limited to about 1 hour to about 1 month. In certain embodiments, the viability and/or activity of the recombinant bacteria in a human host is limited to about 1 hour to about 1 day. In certain embodiments, the viability and/or activity of the recombinant bacteria in a human host is limited to about 1 day to about 1 week. Various methods and mechanism known to control the presence (viability) and/or activity of the bacterial cells in vivo can be utilized. Pharmaceutical compositions according to the present invention, can comprise, in addition to the recombinant bacteria, a pharmaceutically acceptable excipient, carrier, buffer, stabilizer or other materials well known to those skilled in the art. Such materials should be non-toxic and should not interfere with the efficacy of the polypeptides described herein. The precise nature of the carrier or other material can depend on the route of administration.

A bacterium such as a probiotic bacterium can be genetically engineered to express one or more of the FcRn binding polypeptides (optionally with a specific binding moiety) described herein. In an embodiment a vector encoding one or more FcRn binding polypeptides (optionally with a specific binding moiety) can be delivered to a probiotic bacterium such that the probiotic bacterium expresses the one or more FcRn binding polypeptides. In an embodiment, a probiotic bacterium can constantly express and secrete one or more FcRn binding polypeptides (optionally with a specific binding moiety), meaning that external stimulus is not required for expression.

In some embodiments recombinant bacteria are probiotic, commensal, mutualistic or non-pathogenic in humans. In certain embodiments, a recombinant bacterium is capable of reproduction in the body of a mammalian subject or on a mucosal surface of a mammalian subject. A mucosal surface can be, for example, oral mucosa, nasal mucosa, gastrointestinal mucosa, vaginal mucosa, rectal mucosa or urinary bladder mucosa.

A recombinant bacterium can be an auxotroph that is incapable of reproduction on a mucosal surface or in the body of a subject because of a biocontainment strategy, which can prevent the continued reproduction, division, or proliferation of the recombinant bacteria in the mammalian host. Biocontainment can be achieved by, for example, introducing a suicide gene into the bacteria that is kept in an off state by a factor supplied to the bacteria when they are grown in culture, but that is not present in a healthy mammalian body or on the mucosal surface of the mammalian subject. In an example, a recombinant bacterium can be auxotrophic in that it lacks an active essential gene product such that it depends on the presence of the gene product in cell culture medium or in the treated mammalian body. In an embodiment, an essential gene can be inactivated by deletion or replacement of a polynucleotide sequence. An essential gene can be, for example, a thymidylate synthase gene. In this case growth of the bacterium depends on the presence of thymidine and/or thymine. Another example is bacteria that lack an active alanine racemase gene required for growth, rendering their growth dependent on the presence of D-alanine.

In certain embodiments, the viability and/or activity of a recombinant bacterium in a human host is limited to about 1 hour to about 1 month. In certain embodiments, the viability and/or activity of the transgenic bacterium in a human host is limited to about 1 hour to about 1 day. In certain embodiments, the viability and/or activity of the transgenic bacterium in a human host is limited to about 1 day to about 1 week.

In some embodiments a bacterial promoter nucleic acid sequence can be used to control the expression of the one or more FcRn binding polypeptides (optionally with a specific binding moiety). In some embodiments, a recombinant bacterium expresses and secretes the one or more FcRn binding polypeptides (optionally with a specific binding moiety) in response to an external stimulus using an inducible promoter. An external stimulus is a signal external to the bacteria, such as an environmental, biological, or chemical signal to which expression and/or secretion are functionally linked.

In an aspect a recombinant bacterium can be genetically modified to express and secrete one or more FcRn binding polypeptides (optionally with a specific binding moiety). In certain embodiments, the one or more FcRn binding polypeptides (optionally with a specific binding moiety) can be expressed from an exogenous expression cassette comprising a transcribable polynucleotide encoding the one or more FcRn binding polypeptides (optionally with a specific binding moiety). In certain embodiments, the one or more FcRn binding polypeptides (optionally with a specific binding moiety) are expressed from an exogenous expression cassette comprising a transcribable polynucleotide encoding the one or more FcRn binding polypeptides operably linked to one or more expression control sequences and optionally with a specific binding moiety. In certain embodiments, the expression control sequence comprises a constitutive promoter. In certain embodiments, the expression control sequence comprises an inducible promoter. In certain embodiments, the exogenous expression cassette is carried by a plasmid or other vector. In certain embodiments, the exogenous expression cassette is integrated to the bacterial genome.

The delivery of recombinant bacteria can be made according to any method known in the art. According to some embodiments, the recombinant bacteria is encapsulated to allow sustained release and/or to improve the delivery to the treated area, for example to a specific area of the gastrointestinal system.

In certain embodiments, the recombinant bacterium is of the order Lactobacillales. In certain embodiments, the recombinant bacterium is of the family Lactobacillaceae. In certain embodiments, the recombinant bacterium is of the genus Lactobacillus. Bacillus, Bacteroides, Streptococcus, Bifidobacterium, Corynebacterium, Clostridium, Escherichia, Lactococcus, Leuconostoc, Pediococcus, Aerococcus, Carnobacterium, Enterococcus, Oenococcus, Sporolactobacillus, Teragenococcus, Vagococcus, and Weisella. Examples of species that can be used include Lactobacillus acidophilus, Lactobacillus crispatus, Lactobacillus gasseri, Lactobacillus delbreuckii, Lactobacillus rhamnosus, Lactobacillus salivarius, Lactobacillus paracasei, Lactobacillus reuteri, Lactobacillus bulgaricus, Lactobacillus casei, Lactobacillus lactis, Lactobacillus plantarum, Lactobacillus rhamnosus, Bifidobacterium longum, Bifidobacterium breve, Bifidobacterium lactis, Lactobacillus reuterior, Lactobacillus fermentum, Escherichia coli strain Nissle, Escherichia coli strain Symbioflor, and Bacillus subtilis strain 168.

In certain embodiments, the pharmaceutical composition comprising the population of recombinant bacteria can be formulated for mucosal delivery, namely formulated to be suitable for application to a mucosal membrane. The term “mucosal delivery” as used herein refers to the delivery to a mucosal surface, including oral, gastrointestinal, nasal, pulmonary, vaginal, rectal, urethral, sublingual or buccal delivery. In certain embodiments, the composition is formulated for oral delivery. The term “oral delivery” as used herein refers to delivery to, or via, the oral cavity. In certain embodiments, the pharmaceutical composition is formulated as a nutraceutical product. In certain embodiments, the pharmaceutical composition is formulated for rectal delivery. In certain embodiments, the pharmaceutical composition is formulated as a suppository or as an enema. In certain embodiments, the pharmaceutical composition is formulated as a gel, a paste or an ointment. In other embodiments, the pharmaceutical composition is formulated as a liquid, semi liquid or a suspension. In certain embodiments, the pharmaceutical composition is formulated as a tooth paste or as an oral rinse.

Methods of Treatment

The present disclosure provides methods of treatment using the polypeptides e.g., FcRn polypeptides, antibodies containing FcRn binding polypeptides, FcRn binding polypeptides with a specific binding moiety) and/or pharmaceutical compositions of the same. As used herein, the terms “treating”, “treat” or “treatment” include administering polypeptides, antibodies and/or pharmaceutical compositions described herein to thereby reduce or eliminate at least one symptom of a specified disease or condition. As used herein, the term “subject” shall be taken to mean any animal including humans, for example a mammal. Exemplary subjects include but are not limited to humans and non-human primates. For example, the subject is a human.

FcRn engagement can enhance the gut bioavailability and residence time of IV-dosed antibody fragments via FcRn-mediated blood-to-gut transport. Thus, modification of antibody fragments with the FcRn-polypeptide described here can dramatically enhance the impact of these therapeutics against GI indications including, but not limited to, inflammatory bowel disease or recalcitrant Clostridioides difficile infection. Furthermore, FcRn engagement has been shown to enable gut-to-blood transport and can facilitate alternative routes of scFv administration including oral dosing, vectored immunoprophylaxis, or in situ production by engineered commensal microbes, which have can used as delivery vehicles for a host of antibodies and small protein therapeutics.

FcRn polypeptides, antibodies, and/or pharmaceutical compositions thereof of the present disclosure can be useful in the treatment of cancers. Exemplary cancers include cystic and solid tumors, bone and soft tissue tumors, including tumors in anal tissue, bile duct, bladder, blood cells, bowel, brain, breast, carcinoid, cervix, eye, esophagus, head and neck, kidney, larynx, leukemia, liver, lung, lymph nodes, lymphoma, melanoma, mesothelioma, myeloma, ovary, pancreas, penis, prostate, skin, sarcomas, stomach, testes, thyroid, vagina, vulva. Soft tissue tumors include Benign schwannoma Monosomy, Desmoid tumor, lipo-blastoma, lipoma, uterine leiomyoma, clear cell sarcoma, dermatofibrosarcoma, Ewing sarcoma, extraskeletal myxoid chondrosarcoma, liposarcooma myxoid, Alveolar rhabdomyosarcoma and synovial sarcoma. Specific bone tumors include non-ossifying fibroma, unicameral bone cyst, enchondroma, aneurismal bone cyst, osteoblastoma, chondroblastoma, chondromyxofibroma, ossifying fibroma and adamantinoma, Giant cell tumor, fibrous dysplasia, Ewing's sarcoma eosinophilic granuloma, osteosarcoma, chondroma, chondrosarcoma, malignant fibrous histiocytoma and metastatic carcinoma. Leukemias include acute lymphoblastic, acute myeloblastic, chronic lymphocytic and chronic myeloid.

FcRn polypeptides, FcRn binding polypeptides and specific biding moiety, antibodies, and/or pharmaceutical compositions thereof are useful in the treatment and prevention of human viral infections. Examples of viral infections include infections caused by DNA viruses (e.g., Herpes Viruses such as Herpes Simplex viruses; Epstein-Barn virus; Cytomegalovirus; Pox viruses such as Variola (small pox) virus; Hepadnaviruses (e.g., Hepatitis B virus); Papilloma viruses; Adenoviruses); RNA Viruses (e.g., HIV I, II; HTLV I, II; Poliovirus; Hepatitis A; coronaviruses, such as sudden acute respiratory syndrome (SARS); Orthomyxoviruses (e.g., Influenza viruses); Paramyxoviruses (e.g., Measles virus); Rabies virus: Hepatitis C virus), Flaviviruses, Influenza viruses; caliciviruses; rabies viruses, rinderpest viruses, Arena virus, and the like. Moreover, examples of the types of virus-related diseases include but are not limited to: acquired immunodeficiency; hepatitis; gastroenteritis; hemorrhagic diseases; enteritis; carditis; encephalitis; paralysis; bronchiolitis; upper and lower respiratory disease; respiratory papillomatosis; arthritis; disseminated disease, meningitis, mononucleosis.

FcRn polypeptides, antibodies, and/or pharmaceutical compositions thereof are also useful in the treatment of microbial infections including Chiarnydia trachomatis, Listeria sp., Helicobacter pylori, Mycobacterium sp., Mycoplasma sp., Bacillus sp., Salmonella sp., and Shigella sp., E. coli, Clostridium sp.

Compositions and methods are more particularly described below, and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

The amino acid abbreviations used herein are:

| | 1 letter | |
|---|---|---|
| Abbreviation | abbreviation | Amino acid name |
| Ala | A | Alanine |
| Arg | R | Arginine |
| Asn | N | Asparagine |
| Asp | D | Aspartic acid |
| Cys | C | Cysteine |
| Gln | Q | Glutamine |
| Glu | E | Glutamic acid |
| Gly | G | Glycine |
| His | H | Histidine |
| Ile | I | Isoleucine |
| Leu | L | Leucine |
| Lys | K | Lysine |
| Met | M | Methionine |
| Phe | F | Phenylalanine |
| Pro | P | Proline |
| Pyl | O | Pyrrolysine |
| Ser | S | Serine |
| Sec | U | Selenocysteine |
| Thr | T | Threonine |
| Trp | W | Tryptophan |
| Tyr | Y | Tyrosine |
| Val | V | Valine |
| Asx | B | Aspartic acid or Asparagine |
| Glx | Z | Glutamic acid or Glutamine |
| Xaa | X | Any amino acid |

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

### EXAMPLES

### Example 1

Example 1 describes the experimental methods used in the experiments described in Examples 2-7.

Plasmid Construction

All recombinant proteins were expressed using standard methods. The gene encoding the extracellular domain (ECD) of HER2 was reverse transcribed from HEK293T total RNA. The extracellular domain of human FcRn was amplified from a total RNA pool of T84 cells (ATCC CCL-248). All trastuzumab scFv polypeptides, as well as an FcRn-μ2m fusion protein, were fused to the signal peptide of E. coli outer membrane protein A (OmpA) to promote export to the periplasmic space to facilitate proper disulfide formation.

In Silico Structure Prediction

To generate computational models of scFv antibodies modified with Fc- or albumin-mimetic peptides, the amino acid sequence of each variant was input into the Robetta structure prediction web server. In the case of variants modified with cyclic Fc-mimetic peptides, PDB 3M17 was provided as part of the template library for comparative modeling of the peptide domain. Similarly, PDB 1A06 was provided as a potential template for scFvs modified with the albumin-mimetic peptide domain. The models with the lowest estimated angstrom error were selected for further investigation. The Haddock2.2 webserver was then used to simulate the interaction between the FcRn-μ2m complex (PDB 4NOU) and the modeled scFv structures. Interacting residues were restricted to the Fc- or albumin-mimetic peptides on the scFvs and the canonical FcRn-IgG and FcRn-albumin binding interfaces identified in previous crystallography studies. Docking models were selected based on the Haddock best practice guide published at bonvinlab.org/software/bpg/.

Cellular Recycling and Transcytosis Assays

All cell-based experiments used T84 human colon carcinoma cells (ATCC CCL-248). T84 cultures were maintained in 1:1 DMEM/F12 (Corning) supplemented with 10% (v/v) fetal bovine serum (Thermo Fisher) and 1% (v/v) Antibiotic-Antimycotic (Gibco) at 37° C., 5% CO₂. Recycling and transcytosis assays were performed as previously described (Grevys, Foss). Briefly, for recycling experiments, cells were seeded at 2×10⁵ cells/well in 48-well cell culture plates (VWR). Once cells reached confluency (˜3 days), growth media was aspirated, and cells were starved for 1 hour in HBSS at pH 6.0 (Gibco). Antibodies and antibody fragments were diluted to 400 nM in HBSS (pH 6.0) and were incubated with T84 monolayers for 4 hours at 37° C. Antibody solutions were aspirated, and monolayers were washed with cold HBSS at pH 7.4. Cells were then incubated overnight at 37° C. in DMEM/F12 without FBS to facilitate release of internalized antibodies. Antibody release was detected by ELISA and quantified by comparison to a standard curve of the corresponding purified antibody. For competitive recycling assays, experimental antibodies and antibody fragments were biotinylated with NHS-biotin (ApexBio) according to the manufacturer's instructions, then diluted to 400 nM in HBSS (pH 6.0) containing 12 μM unlabeled human IgG or albumin. Release of biotinylated antibody or antibody fragment was detected by ELISA with streptavidin-HRP.

For transcytosis assays, cells were seeded at 1.5×105 cells/well on 0.4 μm PET transwell inserts (Sterlitech) in 24-well tissue culture plates. Culture media was changed every −2 days. Trans-epithelial electrical resistance (TEER) was monitored using a Millicell ERS-2 voltohmmeter (Millipore Sigma). Assays were performed on monolayers exhibiting TEER values of 1000-1500 Q*cm 2. Once the monolayers were polarized, growth media was aspirated and replaced with HBSS at pH 6 (apical chamber) or pH 7.4 (basolateral chamber) for 1 hour to starve cells. Full-length antibodies or antibody fragments were labeled with AFDye 488 NHS Ester (Click Chemistry Tools) at 10-fold molar excess as per manufacturer's instructions. Unreacted dye was removed by desalting on a PD-10 column, and the resulting labeled antibodies were filtered through 0.2 μm PES filters (VWR) before being diluted to 4 μM in HBSS (pH 6) and added to the apical chamber. DMEM/F12 without FBS was added to the basolateral chamber. 50 μL samples were collected from the basolateral chamber after 2 hours of incubation and loaded into a black 384-well plate (4titude). Transcytosed antibodies or antibody fragments were detected by fluorescence measurement (Ex/Em: 485/528) on a BioTek Synergy HT multimode microplate reader. Sample concentration was estimated using a standard curve of purified, fluorescently tagged antibody. Wells that had an obvious leak in the monolayer (informed by abnormally high fluorescence signal in endpoint assay and subsequent verification via visual inspection of monolayer under 20× magnification) were excluded from analysis.

All recombinant proteins were expressed in BL21 DE3 E. coli using a pET24b+ plasmid expression system. The gene encoding the extracellular domain (ECD) of HER2 was reverse transcribed from HEK293T total RNA using primer HER2_RT_2. The membrane-proximal domain was then cloned into the Ndel and Xhol sites of pET24b+, upstream of an in-frame hexahistidine tag (SEQ ID NO: 9) with primers HER2_ECD_Prox_1 and HER2_ECD_Prox_2. All trastuzumab scFv variants, as well as an FcRn-μ2m fusion protein, were fused to the signal peptide of E. coli outer membrane protein A (OmpA) to promote export to the periplasmic space to facilitate proper disulfide formation. The OmpA signal peptide was inserted into the Ndel and BamHI restriction sites of pET24b+ with primers OmpA_Nde_F and OmpA_Bam_R to generate plasmid pET24b+-OmpA. The extracellular domain of human FcRn was amplified from a total RNA pool of T84 cells (ATCC CCL-248) using primers hFcRn_F and hFcRn_R and fused to a synthetic gene encoding human p2m codon-optimized for expression in E. coli (GenScript), connected by a (Gly₄Ser)₃ linker domain (SEQ ID NO: 6) using primers hFcRn_hB2F and hB2m_hFcRnR to generate complimentary overlaps and hB2m-Omp-Bam-F and hFcRn_R to amplify the fused genes. This fusion protein was then cloned into the BamHI and Xhol restriction sites of pET24b+-OmpA, upstream of the in-frame hexahistidine tag (SEQ ID NO: 9) and downstream of the OmpA signal peptide with primers hB2m-Omp-Bam-F and thFcRn_R. Trastuzumab scFvs were modified with FcRn-binding peptides using whole-plasmid amplification and blunt-end ligation in pUC57. These fusions were then cloned into the BamHI and Xhol restriction sites of pET24b+-OmpA with a triple stop codon upstream of the hexahistidine tag (SEQ ID NO: 9). All plasmids were verified by DNA sequencing.

Recombinant Protein Expression and Purification

HER2 ECD was expressed and purified as previously described, with minor modifications. Briefly, BL21 (DE3) E. coli harboring pET24b+-HER2 ECD were grown in lysogeny broth (LB) at 37° C. to an OD600 of approximately 0.6. Protein expression was induced by addition of isopropyl p-d-1-thiogalactopyranoside (IPTG) to a final concentration of 0.1 mM, followed by incubation at 37° C. overnight (14 hours). Cells were pelleted and lysed by sonication in 1× phosphate buffered saline (PBS)/1% (v/v) Triton X100. The insoluble fraction was solubilized in 8 M urea overnight (14 hours). Recombinant HER2 ECD was isolated using Ni-NTA affinity chromatography on an AKTA FPLC (Amersham Biosci). Prior to elution, on-column re-folding was performed by titration from 8 M to 0 M urea. Impurities were removed by washing with 40 mM imidazole, followed by elution using 500 mM imidazole. Eluted protein was buffer exchanged into 1× PBS using PD-10 desalting columns (Millipore Sigma), and purity was evaluated by SDS-PAGE. To purify FcRn-μ2m fusion protein, BL21 (DE3) E. coli harboring pET24b+-OmpA-μ2m-FcRn were grown and induced with IPTG as above, followed by a 30-minute incubation at 42° C. and overnight (14 hours) incubation at 22° C. Cells were pelleted and lysed as above. FcRn-μ2m was isolated from the soluble fraction using Ni-NTA affinity chromatography and processed as above. For all trastuzumab scFv variants, BL21(DE3) E. coli cultures harboring pET24b+-OmpA-scFv were grown and induced as above, followed by incubation at 37 degrees C. overnight (14 hours). Protein was then purified by protein L affinity chromatography from the soluble fraction of the cell lysate. All purified proteins were stored in sterile 1× PBS supplemented with 0.1 mM phenylmethylsulfonyl fluoride (PMSF) protease inhibitor at 4° C. for short-term storage (<2 weeks), or at −20° C. with 25% (v/v) glycerol for long-term storage (2 weeks −1 year).

In Silico Structure Prediction

To generate computational models of scFv antibodies modified with Fc- or albumin-mimetic peptides, the amino acid sequence of each variant was input into the Robetta structure prediction web server. In the case of variants modified with cyclic Fc-mimetic peptides, PDB 3M17 was provided as part of the template library for comparative modeling of the peptide domain. Similarly, PDB 1A06 was provided as a potential template for scFvs modified with the albumin-mimetic peptide domain. The models with the lowest estimated angstrom error were selected for further investigation. The Haddock2.2 webserver was then used to simulate the interaction between the FcRn-μ2m complex (PDB 4NOU) and the modeled scFv structures. Interacting residues were restricted to the Fc- or albumin-mimetic peptides on the scFvs and the canonical FcRn-IgG and FcRn-albumin binding interfaces identified in previous crystallography studies. Docking models were selected based on the Haddock best practice guide published at bonvinlab.org/software/bpg/.

Binding ELISA

Purified HER2 ECD or FcRn-μ2m was coated at 2 μg/mL on Immulon HB microtitier plates (Immulon) overnight (14 hours) at 4° C. After warming to room temperature (RT), plates were blocked with 3% (w/v) bovine serum albumin (BSA) in PBS+0.05% (v/v) Tween 20 (PBS-T) at pH 7.4 or 5.5 for one hour at RT. Full-length antibodies or antibody fragments were then added in pH 7.4 or pH 5.5 blocking solution and plates were incubated for 1-2 hours at RT. After three PBS-T washes, bound antibody was detected by incubation with a 1:5000 dilution of protein L-HRP (VWR) for 1 hour, followed by development with o-phenylenediamene (OPD) (BioBasic) as per manufacturer's instructions. For competitive ELISAs, antibodies were biotinylated with NHS-biotin (ApexBio) according to the manufacturer's instructions, then diluted in PBS-T+3% (w/v) BSA with human serum albumin (Sigma-Aldrich) or unlabeled bulk IgG from human serum (Millipore Sigma) at 10× molar excess over the experimental antibody. Biotinylated antibodies were detected with 1:10000 streptavidin-HRP (Abcam) and OPD as above. Absorbance was measured at 450 nm (A450), and the A450 of blank wells was subtracted before analysis. All assays were performed in triplicate, and statistical analysis was performed using GraphPad Prism.

Cellular Recycling and Transcytosis Assays

All cell-based experiments used T84 human colon carcinoma cells (ATCC CCL-248). T84 cultures were maintained in 1:1 DMEM/F12 (Corning) supplemented with 10% (v/v) fetal bovine serum (Thermo Fisher) and 1% (v/v) Antibiotic-Antimycotic (Gibco) at 37° C., 5% CO₂. Recycling and transcytosis assays were performed as previously described (Grevys, Foss). Briefly, for recycling experiments, cells were seeded at 2×10⁵ cells/well in 48-well cell culture plates (VWR). Once cells reached confluency (˜3 days), growth media was aspirated, and cells were starved for 1 hour in HBSS at pH 6.0 (Gibco). Antibodies and antibody fragments were diluted to 400 nM in HBSS (pH 6.0) and were incubated with T84 monolayers for 4 hours at 37° C. Antibody solutions were aspirated, and monolayers were washed with cold HBSS at pH 7.4. Cells were then incubated overnight at 37° C. in DMEM/F12 without FBS to facilitate release of internalized antibodies. Antibody release was detected by ELISA and quantified by comparison to a standard curve of the corresponding purified antibody. For competitive recycling assays, experimental antibodies and antibody fragments were biotinylated with NHS-biotin (ApexBio) according to the manufacturer's instructions, then diluted to 400 nM in HBSS (pH 6.0) containing 12 μM unlabeled human IgG or albumin. Release of biotinylated antibody or antibody fragment was detected by ELISA with streptavidin-HRP as above.

For transcytosis assays, cells were seeded at 1.5×10⁵ cells/well on 0.4 μm PET transwell inserts (Sterlitech) in 24-well tissue culture plates. Culture media was changed every −2 days. Trans-epithelial electrical resistance (TEER) was monitored using a Millicell ERS-2 voltohmmeter (Millipore Sigma). Assays were performed on monolayers exhibiting TEER values of 1000-1500 Q*cm 2. Once the monolayers polarized, growth media was aspirated and replaced with HBSS at pH 6 (apical chamber) or pH 7.4 (basolateral chamber) for 1 hour to starve cells. Full-length antibodies or antibody fragments were labeled with AFDye 488 NHS Ester (Click Chemistry Tools) at 10-fold molar excess as per manufacturer's instructions. Unreacted dye was removed by desalting on a PD-10 column, and the resulting labeled antibodies were filtered through 0.2 μm PES filters (VWR) before being diluted to 4 μM in HBSS (pH 6) and added to the apical chamber. DMEM/F12 without FBS was added to the basolateral chamber. 50 μL samples were collected from the basolateral chamber after 2 hours of incubation and loaded into a black 384-well plate (4titude). Transcytosed antibodies or antibody fragments were detected by fluorescence measurement (Ex/Em: 485/528) on a BioTek Synergy HT multimode microplate reader. Sample concentration was estimated using a standard curve of purified, fluorescently tagged antibody. Wells that had an obvious leak in the monolayer (informed by abnormally high fluorescence signal in endpoint assay and subsequent verification via visual inspection of monolayer under 20× magnification) were excluded from analysis.

### Example 2

Modification of trastuzumab scFv with FcRn polypeptides

Trastuzumab was selected to evaluate FcRn polypeptides. Trastuzumab scFvs were generated in the V_{L}-V_{H} orientation with a standard (Gly₄Ser)₃ linker (SEQ ID NO: 6) i.e., (GGGGS)₃. (SEQ ID NO:6) Previous studies using polypeptides to enable the trans-epithelial transcytosis of larger proteins identified a series of polypeptides with 16 amino-acid peptides that bind FcRn in a pH-dependent fashion (Sockolosky JT et al. PNAS. 2012 Oct. 2; 109(40):16095-100 and Mezo AR, et al. (2008) PNAS 105:2337-2342). The polypeptides have a mostly conserved consensus sequence, with two important variations namely 1) replacement of Val4 and Ala14 with cysteines adds a disulfide bond and promotes a cyclic (Cyc) rather than linear (Lin) peptide conformation, and 2) replacement of Tyr12 in the parental peptide with a His residue (Y12H) enhances pH-dependent binding to FcRn. The FcRn polypeptides.

TABLE 1
FcRn polypeptides

| Identifier | Sequence | SEQ ID NO: |
|---|---|---|
| Cyc | QRFCTGHFGGLYPCNG | 2 |
| Cyc Y12H | QRFCTGHFGGLHPCNG | 3 |
| Lin | QRFVTGHFGGLYPANG | 4 |
| Lin Y12H | QRFVTGHFGGLHPANG | 5 |

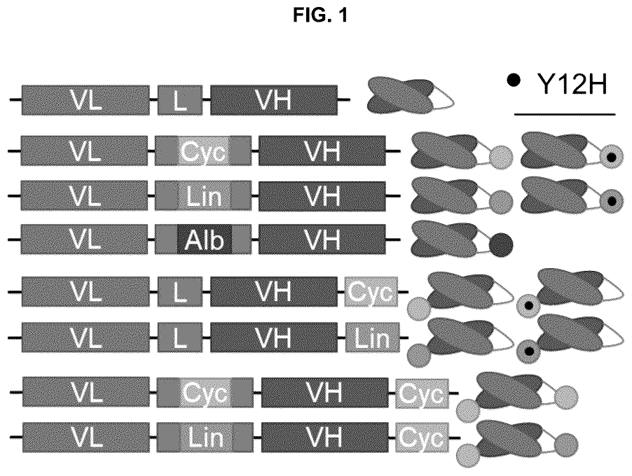

HER2-targeting scFv variants were prepared and modified with each of these four architectures (Cyc, CycY12H, Lin, LinY12H) in each of three orientations: as C-terminal extensions, as part of the linker connecting the V_{H} and V_{L} domains, or as both, resulting in a collection of 12 modified variants (FIG. 1). Only 4 double-peptide variants were generated as follows: V_{L}-Cyc-V_{H}-Cyc, V_{L}-Lin-V_{H}-Cyc, V_{L}-CycY12H-V_{H}-Lin, and V_{L}-Lin-V_{H}-CycY12H.

Building on the evidence that small peptide domains mimicking IgG Fc can enable FcRn-mediated interaction with fused proteins, the approach was expanded by generating an alternative FcRn binding polypeptide with orthogonal binding site specificity. Human serum albumin interacts extensively with FcRn, which promotes extended serum half-life, and albumin is also robustly transported across polarized epithelia by FcRn. Further, albumin binds at a distinct site on FcRn and does not compete with IgG for binding. The human albumin crystal structure (PDB 4NOU) was analyzed, and a 14-amino acid stretch at the albumin-FcRn binding interface (Tyr497—His510) with several similarities to the FcRn polypeptides, including a core hydrophobic residue (Phe509, 13, and 8 in albumin, albumin peptide, and FcRn polypeptides, respectively) and a histidine in close proximity to acidic residues on FcRn. This peptide (FcRn polypeptide-Albumin) was grafted into the linker of the trastuzumab-derived scFv, flanked by single-repeat of GGGGS linker (SEQ ID NO:7) (see FIG. 1).

### Example 3

Computational modeling of scFv-FcRn binding polypeptide structure and binding to FcRn

To explore the impact of the FcRn binding polypeptides on the structure of the scFvs, the Robetta webserver was used to generate predicted three-dimensional structures of scFv-FcRn polypeptide, which were then compared to the unmodified trastuzumab scFv (PDB 6J71) (Wang, Z., et al. (2019) Acta Crystallogr. Sect. D Struct. Biol. 75, 554-563). The results revealed minimal differences between the unmodified and modified structures, suggesting that the peptides do not impact the folding or overall structure of the scFvs. Model predictions with the lowest angstrom error estimate were used to perform molecular docking simulations with FcRn in its native heterodimeric conformation with (32m, using the HADDOCK2.2 webserver and compared the resulting models to the native Fc-FcRn complex. Docked models revealed several key interactions between the canonical FcRn-Fc interface and scFv-FcRn polypeptides. Notably, histidine residues in both the intra-linker (His121) and C-terminal (His254) Cyc and Lin FcRn binding polypeptides make close contacts with FcRn residues Glu116 and Asp121, as well as Asp130, which is also involved in the interaction with full-length IgG. Furthermore, in docked models of scFv-FcRn binding polypeptides with C-terminal or intra-linker FcRn binding polypeptides containing both the cyclizing cysteine mutations (peptide positions V4C/A14C of SEQ ID NO:2 and SEQ ID NO:3) and the acidifying Y12H mutation (His126 [linker] or 259 [C-term]), both of the peptide histidines (His121 and 126 [linker] or His254 and 259 [C-term]) form close contacts with acidic FcRn residues, as expected. Conversely, models of linear scFv-FcRn binding polypeptide revealed that the Y12H mutation leads to a kinked peptide conformation, increasing the distance between His121 or 254 and acidic FcRn residues Glu116, Asp121, and Asp130, and decreasing predicted binding strength. Docking simulations of FcRn with an FcRn binding polypeptide and Albumin-modified scFv also revealed key similarities with the native human albumin-FcRn interaction, including a close contact between Asp231 on FcRn with His510 on albumin that is mimicked by His126 on the FcRn polypeptide-Alb-modified scFv as well as a hydrophobic interaction between FcRn Trp53 and Phe507 and 509 on albumin which is mimicked by Phe118 on the FcRn Polypeptide-Albumin-modified scFv.

### Example 4

Protein expression, purification, and characterization

scFv-FcRn binding polypeptides were introduced into the periplasm of E. coli to facilitate proper folding and formation of internal disulfide bonds. Protein yields for unmodified scFv and all FcRn-scFv polypeptides were ˜3-5 mg/L of E. coli culture, suggesting that the FcRn binding polypeptides do not negatively impact scFv folding or stability. SDS-PAGE analysis revealed expected shifts in molecular weight for all FcRn-scFv polypeptides compared to the unmodified scFv. The ability of all FcRn-scFv polypeptides to bind their target antigen, HER2, by ELISA using a truncated HER2 extracellular domain expressed and purified from E. coli. Estimated affinity constants for each scFv variant were calculated by applying a saturation binding kinetics model to the ELISA data (GraphPad Prism 9). The incorporation of FcRn binding polypeptide into the scFvs did not impact target antigen binding in these constructs, as estimated affinities of the variants were not significantly different from either the unmodified scFv or full-length control (Table 2). In Table 2, ±denotes standard error of triplicate samples.

TABLE 2
Estimated KD for binding of modified scFvs to HER2

| | Calculated |
|---|---|
| IgG/scFv | K_{D} (nM)^{a} |
| Trastuzumab IgG | 1.4 ± 0.3 |
| Unmodified scFv | 5.6 ± 2.8 |
| V_{L}-Cyc-V_{H} | 8.5 ± 10 |
| V_{L}-V_{H}-Cyc | 1.6 ± 0.8 |
| V_{L}-Lin-V_{H} | 6.1 ± 1.4 |
| V_{L}-V_{H}-Lin | 2.5 ± 1.9 |
| V_{L}-Cyc(Y12H)-V_{H} | 2.3 ± 1.5 |
| V_{L}-Lin(Y12H)-V_{H} | 2.4 ± 1.2 |
| V_{L}-V_{H}-Lin(Y12H) | 2.9 ± 1.3 |
| V_{L}-V_{H}-Cyc(Y12H) | 0.7 ± 0.3 |
| V_{L}-Cyc-V_{H}-Cyc | 2.7 ± 4 |
| V_{L}-Lin-V_{H}-CycY12H | 6.4 ± 1 |
| V_{L}-CycY12H-V_{H}-Lin | 9.3 ± 2 |
| V_{L}-Alb-V_{H} | 0.8 ± 0.2 |

### Example 5

Binding of FcRn-scFv Polypeptides to FcRn

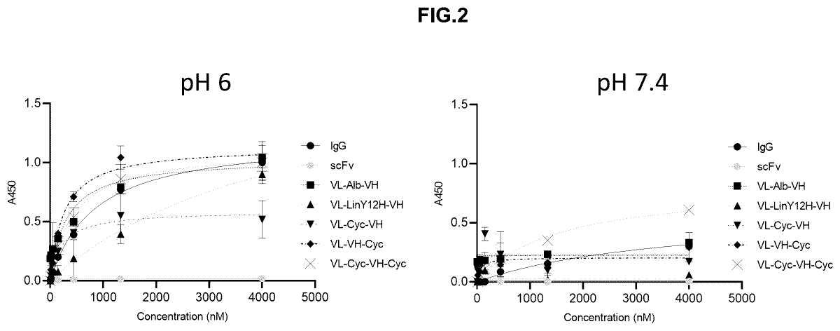

To evaluate the FcRn-binding properties of the modified scFvs, a fusion of β2m to the extracellular domain of FcRn was constructed. The FcRn-μ2m complex bound albumin and IgG Fc in a pH-dependent fashion, with micromolar binding affinity at pH<6 and minimal binding at pH >7. FcRn polypeptide-Fc recapitulates this pH-dependent interaction when fused to a fluorescent reporter protein as previously shown. To generate the FcRn- μ2m complex, an E. coli codon-optimized human μ2m gene was fused to the extracellular domain of human FcRn, connected by a flexible (Gly₄Ser)₃ linker (SEQ ID NO: 6) and expressed and purified the protein from E. coli. scFv binding to FcRn- μ2m was studied by ELISA at both pH 6 and pH 7.4. ELISA analysis of scFvs modified with cyclic FcRn polypeptides-Fc, linear FcRn binding polypeptides-Fc, or FcRn binding polypeptides-Alb behaved similarly to full-length IgG or albumin (FIG. 2), with estimated KD values ranging from 0.3-1.3 μM at pH 6 (Table 3). In Table 3, ±denotes standard error of triplicate samples.

TABLE 3
Estimated KD for binding of modified scFv to FcRn at pH 6

| | Calculated |
|---|---|
| IgG/scFv | K_{D} (μM)^{a} |
| Trastuzumab IgG | 0.9 ± 0.1 |
| Unmodified scFv | ND |
| V_{L}-Cyc-V_{H} | 0.2 ± 0.1 |
| V_{L}-V_{H}-Cyc | 0.3 ± 0.1 |
| V_{L}-Lin-V_{H} | 0.3 ± 0.1 |
| V_{L}-V_{H}-Lin | 0.3 ± 0.1 |
| V_{L}-Cyc(Y12H)-V_{H} | 1.2 ± 0.1 |
| V_{L}-V_{H}-Cyc(Y12H) | 0.3 ± 0.1 |
| V_{L}-Lin(Y12H)-V_{H} | 0.05 ± 0.04 |
| V_{L}-V_{H}-Lin(Y12H) | 0.1 ± 0.1 |
| V_{L}-Cyc-V_{H}-Cyc | 0.4 ± 0.1 |
| V_{L}-Lin-V_{H}-CycY12H | 0.4 ± 0.04 |
| V_{L}-CycY12H-V_{H}-Lin | 0.1 ± 0.03 |
| V_{L}-Alb-V_{H} | 0.3 ± 0.1 |
| Serum Albumin | 0.3 ± 0.03 |

Binding affinity at pH 7.4 was too low to be detectable in this assay. Binding of unmodified scFv to FcRn was also undetectable, at either pH tested, indicating that the interaction of the FcRn binding polypeptide scFvs with FcRn was indeed mediated by their peptide modifications. Neither the acidifying Y12H mutation nor the addition of a second FcRn binding peptide significantly impacted binding at pH 6. However, incorporation of the Y12H mutation resulted in decreased binding at neutral pH. This finding is the expected outcome of replacing tyrosine, which can readily form a hydrogen bond with FcRn, with a pH-titratable histidine that only forms a salt bridge when protonated at low pH.

### Example 6

FcRn binding polypeptide modifications enable FcRn-mediated recycling in T84 cells

Following cellular uptake, albumin and IgG are protected from degradation via pH-dependent binding to FcRn in acidified endosomes and are then trafficked and released either at the surface where they entered (recycling) or at the opposite membrane (transcytosis). The recycling pathway is a key driver of the characteristically long serum half-lives of IgG and albumin. Mimicking such interactions is expected to lead to increased persistence of FcRn-engaging biotherapeutics e.g., therapeutic antibodies in the bloodstream via interactions with FcRn-expressing vascular endothelial cells. Additionally, in pharmacodynamically challenging niches such as the gut lumen, where intestinal epithelial cells express FcRn but intravenously administered biotherapeutics do not accumulate to high levels, FcRn engagement could support sustained bioavailability of these drugs following oral dosing or direct gastrointestinal delivery.

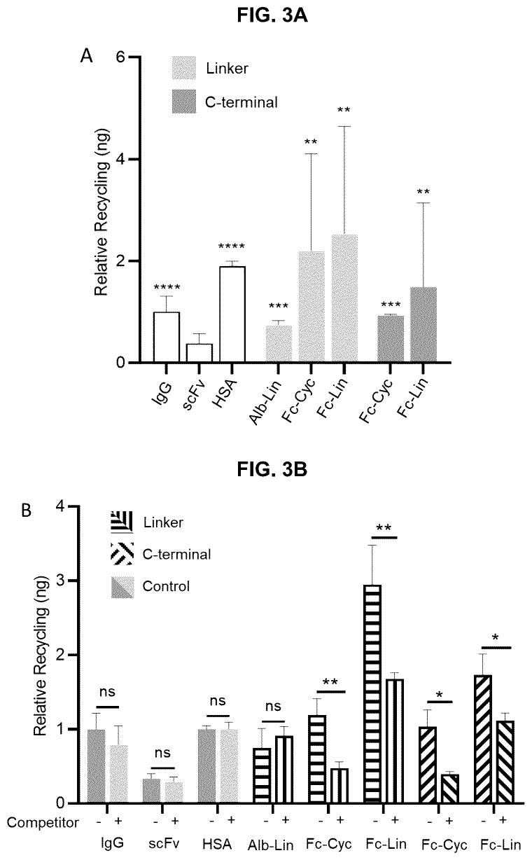

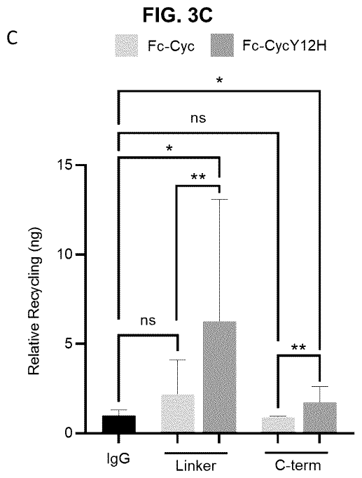

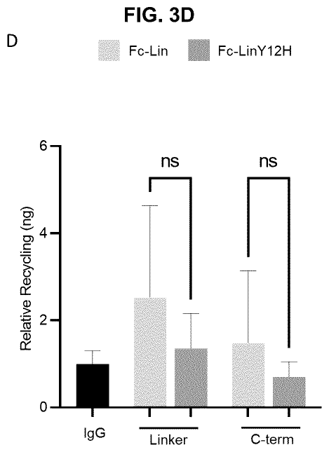

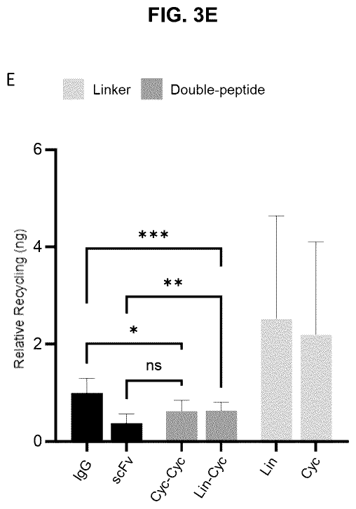

To evaluate FcRn-mediated recycling of FcRn-scFv polypeptides, a cell-based assay was utilized for use with T84 human colorectal carcinoma cells, which endogenously express FcRn. T84 monolayers were incubated with the following five scFvs, each modified with a single peptide: V_{L}-Alb-V_{H}, V_{L}-Cyc-V_{H}, V_{L}-V_{H}-Cyc, V_{L}-Lin-V_{H}, or V_{L}-V_{H}-Lin; along with unmodified scFv and full-length IgG controls. All samples were incubated at pH 5.5 to promote FcRn-mediated retention upon internalization, then washed away non-internalized antibody and incubated overnight at pH 7.4 to allow internalized antibody (protected from endosomal degradation via binding to FcRn) to be recycled and released into the neutral-pH media, which is non-permissive for FcRn binding. All FcRn-scFv polypeptides tested, regardless of FcRn binding polypeptide position (linker or C-terminal), were recycled at least 2-fold more efficiently than unmodified scFv (FIG. 3A), indicating that enhanced scFv recycling is mediated by the FcRn-binding peptides. Next, to verify that the observed recycling was FcRn-specific, the recycling assay was repeated in the presence of a 30-fold molar excess of competing human IgG or albumin. Recycling of all four modified scFvs tested was reduced by 35-60% compared to control samples without competitor added (FIG. 3B), supporting the conclusion that recycling of these constructs is FcRn-dependent. Interestingly, similar reductions were not observed for FcRn polypeptide-Alb-modified scFv or full-length IgG, which can be due to differences in FcRn binding stoichiometry. Because full-length IgG can occupy two FcRn molecules while FcRn polypeptide-modified scFvs can only occupy one, each molar equivalent of full-length IgG could displace two scFvs but only one IgG, leading to more dramatic reductions in recycling for scFvs compared to IgG. Similarly, albumin and FcRn polypeptide-Alb-modified scFvs both bind FcRn in 1:1 stoichiometry. Thus, a higher molar excess of competitor can be required to significantly reduce recycling for IgG, albumin, or FcRn binding polypeptide-Alb-scFv. Competition with the orthogonal full-length proteins albumin or IgG did not significantly impact recycling activity of scFvs modified with FcRn binding polypeptide-Fc or FcRn binding polypeptide-Alb, respectively.

The impact of the Y12H acidifying mutation on FcRn-mediated recycling of scFvs modified with C-terminal or intra-linker FcRn binding polypeptides was tested. Significantly increased recycling of cyclic peptide-modified Y12H scFvs, at levels 2- to 7-fold higher than full-length IgG (FIG. 3C) was observed. The Y12H mutation did not, however, significantly impact recycling of linear FcRn binding polypeptide-Fc variants (FIG. 3D). Finally, the impact on recycling activity of incorporating cyclic or linear FcRn binding polypeptides at both the linker and C-terminal positions within the same construct was reviewed and moderate increases in activity compared to unmodified scFv, but only 60-70% that of the full-length IgG control (FIG. 3E) were observed.

Structural and functional variations arising from peptide position (C-terminal vs. intra-linker), conformation (cyclic vs. linear), or pH sensitivity (parental vs. Y12H mutant) impacted FcRn-mediated activity of the fusion proteins, but not equally so. For example, peptide conformation or position alone does not significantly impact recycling activity; whether cyclic or linear, C-terminal or intra-linker, all peptides lacking the Y12H mutation failed to increase recycling efficiency more than ˜1-2 fold over unmodified scFvs. However, the acidifying Y12H mutation significantly increased recycling activity over unmodified scFvs by 7- and 4-fold when incorporated at either the intra-linker or C-terminal position, respectively, but only with cyclic peptides. In contrast, when incorporated into linear peptides, the Y12H mutation decreased recycling activity, independent of peptide position. Similar results were observed for FcRn-mediated transcytosis of peptide-modified scFvs, with the Y12H mutation leading to a 4-fold increase in activity when added to the V_{L}-Cyc-V_{H} variant and a 5-fold decrease when added to the V_{L}-V_{H}-Lin variant.

In silico structure studies provide insight into the differing effects of the acidifying mutation on the cyclic versus linear FcRn binding polypeptide-Fc. The histidine residues discussed herein fall at positions 7 and 12 of each peptide, which correspond to positions 121 and 126 for scFvs modified with an intra-linker peptide and positions 254 and 259 for scFvs modified with a C-terminal peptide. For both the intra-linker and C-terminal cyclic peptides, the histidine residue(s) of the Cyc (His7) and CycY12H (His7/His12) mutant are predicted to be outward-facing and readily available to interact with FcRn residues Asp110 and Glu105 when protonated. It is thus likely that the enhanced recycling and transcytosis activity observed with CycY12H FcRn binding polypeptide-Fc is due to the additional histidine residue strengthening the interaction with FcRn at pH 6 and increasing scFv salvage within endocytic vesicles. The inverse is true of the linear FcRn binding polypeptide-Fc; energy-minimized crystal structures and docking simulations predict an interaction between FcRn Asp110 and His7 of the non-Y12H mutant, whereas introduction of Y12H leads to a kinked conformation, resulting in both histidines being inaccessible to FcRn.

### Example 7

FcRn binding polypeptide modifications enable FcRn-mediated transcytosis in T84 cells

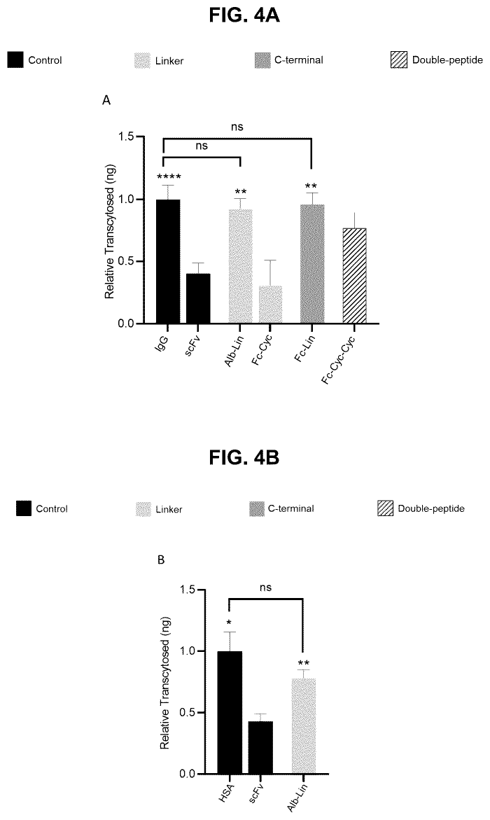

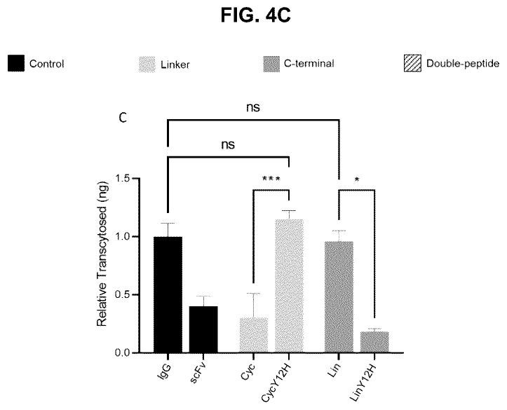

In addition to FcRn-mediated recycling, IgG and albumin internalized by polarized epithelial and endothelial cells can undergo FcRn-mediated transcytosis, whereby the FcRn-bound proteins are trafficked to the opposite membrane (e.g., apical to basolateral), passing through the cell to cross the otherwise impermeable cell barrier. Promoting FcRn-mediated transcytosis of biotherapeutics could improve GI bioavailability of systemically administered drugs via blood-to-gut transport or could enable orally delivered drugs to access the systemic circulation and target non-GI conditions. An established cell-based assay to quantify FcRn-mediated transport of peptide-modified scFvs across polarized T84 epithelial cells grown as monolayers on specialized transcytosis inserts was used. IgG or scFv variants were added to the apical chamber at pH 5.5 and samples were collected from the basolateral chamber two hours later to quantify transport. For these experiments, FcRn polypeptide-Alb as well as the three FcRn binding polypeptides that performed best in recycling assays, within each of the following categories: single cyclic (V_{L}-Cyc-V_{H}), single linear (V_{L}-V_{H}-Lin), and dual peptide (V_{L}-Cyc-V_{H}-Cyc). Of these, V_{L}-Alb-V_{H} and V_{L}-V_{H}-Lin were transported at levels equal to full-length IgG and 2-3-fold higher than unmodified scFv (FIG. 4A). Similarly, FcRn binding polypeptides-Alb-modified scFvs were transported at ˜80% the level of full-length human serum albumin, and 2 times that of the unmodified scFv control (FIG. 4B).

Next, the impact of the Y12H mutation on FcRn-mediated transcytosis of V_{L}-V_{H}-Lin and V_{L}-Cyc-V_{H} was evaluated (FIG. 4C). As in the recycling assays, the Y12H mutation enhanced activity of V_{L}-Cyc-V_{H}, resulting in a nearly 4-fold increase in transport with the mutation versus without, and a 20% increase in transport over V_{L}-V_{H}-Lin. In contrast, transcytosis decreased by >5-fold for V_{L}-V_{H}-Lin upon addition of the Y12H mutation. As with the recycling assays, the computational docking simulations provide insight into these results. Specifically, Cyc-Y12H FcRn binding polypeptide-Fc likely swaps a hydrogen-bonding tyrosine with a pH-titratable histidine, gaining a pH-dependent salt-bridge with FcRn and thus enhanced binding at low pH and decreased binding at neutral pH compared to the non-Y12H variant. Meanwhile, the conformational changes predicted for linear Y12H mutant peptides described in the computational docking studies above likely result in decreased FcRn binding and, subsequently, decreased FcRn-mediated transcytosis activity.

### Example 8

Single-domain antibodies (sdAbs) were designed and tested for activity. aTcdA is sdAb A20.1, which is specifically binds toxin A, TcdA from Clostridioides difficile (see Hussack et al. Journal of Biol. Chem. 2011 Mar. 18; 286(11):8961-76. aTcdA is sdAb A20.1 with no alteration, which is used as a control. NAlb-aTcdA is sdAb A20.1 with SEQ ID NO:8 (YVPKEFNAETFTFH) on the N-terminus of aTcdA. NcYaTcdA is sdAb A20.1 having cyclic SEQ ID NO:3 at the N-terminus. NLaTcdA is sdAb A20.1 with linear SEQ ID NO:4 at the N-terminus.

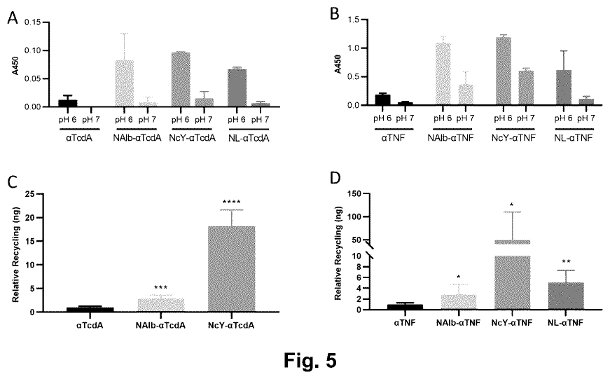

αTNF is a single domain antibody that binds tumor necrosis factor (TNF, inflammatory marker) and is described by Biernaert et al., 2017, Front. Immunol, doi: 10.3389/fimmu.2017.00867. αTNF is a single domain antibody with no alteration, which is used as a control. NAlb-αTNF is αTNF with SEQ ID NO:8 on the N-terminus. NcYaTNF is αTNF having cyclic SEQ ID NO:3 at the N-terminus NLaTNF is αTNF with linear SEQ ID NO:4 at the N-terminus. FIG. 5 panels A and B show binding of these constructs to FcRn at pH 6 and pH 7.4. FIG. 5 panels C and D show FcRn-mediated recycling activity of the constructs.

### Example 9

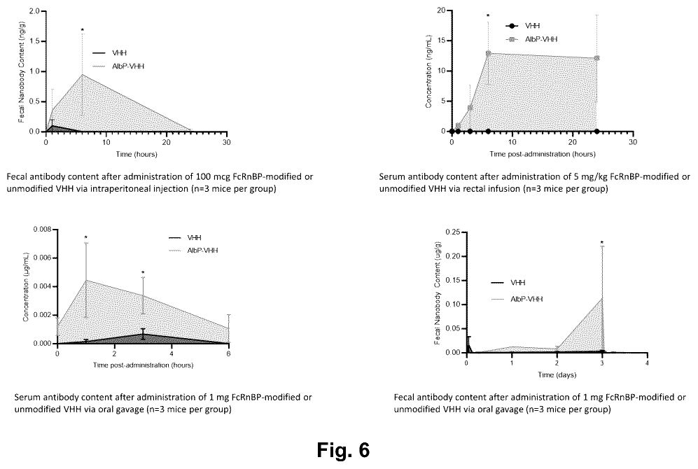

A20.1 sdAb fused to albumin-mimicking peptide (SEQ ID NO:8) (AlbP-V_{HH}) or A20.1 with no modification (V_{HH}) was delivered interperitoneally or by rectal infusion to transgenic mice. These are in vivo data showing functional engagement with FcRn in transgenic mice expressing human FcRn (B6.Cg-Fcgrttm1Dcr Tg(FCGRT)32Dcr/DcrJ, or “Tg32” mice). FIG. 6, upper left panel shows fecal antibody content after administration of 100 mcg FcRnBP-modified (AlbP-V_{HH}) or unmodified V_{HH} via intraperitoneal injection (n=3 mice per group). FIG. 6 upper right panel shows serum antibody content after administration of 5 mg/kg FcRnBP-modified or unmodified V_{HH} via rectal infusion (n=3 mice per group). Accumulation of the antibodies in serum occurs only if the antibodies are transcytosed into the blood stream. FIG. 6 lower right panel shows serum antibody content after administration of 1 mg FcRnBP-modified or unmodified V_{HH} via oral gavage (n=3 mice per group). FIG. 6 lower left panel shows fecal antibody content after administration of 1 mg FcRnBP-modified or unmodified V_{HH} via oral gavage (n=3 mice per group).

1. An isolated polypeptide comprising a neonatal Fc receptor (FcRn) binding polypeptide comprising an amino acid sequence set forth in QRFX₁TGHFGGLX₂PX₃NG (SEQ ID NO:1) or YVPKEFNAETFTFH (SEQ ID NO:8) and a specific binding moiety.

2. The isolated polypeptide of claim 1, wherein the specific binding moiety comprises a V H domain, a V H domain and a V L domain, an antibody fragment, or a nanobody.

3. The isolated polypeptide of claim 1, wherein the FcRn binding polypeptide comprises an amino acid sequence as set forth in SEQ ID NO:2, 3, 4, or 5.

4. An isolated polypeptide comprising, from an N-terminus to a C-terminus:
(a) an antibody light chain variable region (V L) domain, a first neonatal Fc receptor (FcRn) binding polypeptide comprising an amino acid sequence set forth in QRFX₁TGHFGGLX₂PX₃NG (SEQ ID NO:1), and an antibody heavy chain variable region (V H) domain; or
(b) an antibody light chain variable region (V L) domain, a first FcRn binding polypeptide comprising an amino acid sequence set forth in QRFX₁TGHFGGLX₂PX₃NG (SEQ ID NO:1), an antibody heavy chain variable region (V H) domain; and a second FcRn binding polypeptide comprising an amino acid sequence set forth in QRFX₁TGHFGGLX₂PX₃NG (SEQ ID NO:1); or
(c) an antibody light chain variable region (V L) domain, a linker, an antibody heavy chain variable region (V H) domain; and a first FcRn binding polypeptide comprising an amino acid sequence set forth in QRFX₁TGHFGGLX₂PX₃NG (SEQ ID NO:1), or
(d) an antibody light chain variable region (V_{L}) domain, a first FcRn binding polypeptide comprising an amino acid sequence set forth in YVPKEFNAETFTFH (SEQ ID NO:8) and an antibody heavy chain variable region (V_{H}) domain.

5. The isolated polypeptide of claim 4, wherein the first FcRn binding polypeptide comprises an amino acid sequence as set forth in SEQ ID NO:2, 3, 4, or 5.

6. The isolated polypeptide of claim 4, wherein the second FcRn binding polypeptide comprises an amino acid sequence as set forth in SEQ ID NO:2, 3, 4, or 5.

7. The isolated polypeptide of claim 4, wherein;
(a) the first FcRn binding polypeptide comprises an amino acid sequence as set forth in SEQ ID NO:2 and the second FcRn binding polypeptide comprises an amino acid sequence as set forth in SEQ ID NO:2;
(b) the first FcRn binding polypeptide comprises an amino acid sequence as set forth in SEQ ID NO:2 and the second FcRn binding polypeptide comprises an amino acid sequence as set forth in SEQ ID NO:3;
(c) the first FcRn binding polypeptide comprises an amino acid sequence as set forth in SEQ ID NO:2 and the second FcRn binding polypeptide comprises an amino acid sequence as set forth in SEQ ID NO:4;
(d) the first FcRn binding polypeptide comprises an amino acid sequence as set forth in SEQ ID NO:2 and the second FcRn binding polypeptide comprises an amino acid sequence as set forth in SEQ ID NO:5;
(e) the first FcRn binding polypeptide comprises an amino acid sequence as set forth in SEQ ID NO:3 and the second FcRn binding polypeptide comprises an amino acid sequence as set forth in SEQ ID NO:2;
(f) the first FcRn binding polypeptide comprises an amino acid sequence as set forth in SEQ ID NO:3 and the second FcRn binding polypeptide comprises an amino acid sequence as set forth in SEQ ID NO:3;
(g) the first FcRn binding polypeptide comprises an amino acid sequence as set forth in SEQ ID NO:3 and the second FcRn binding polypeptide comprises an amino acid sequence as set forth in SEQ ID NO:4;
(h) the first FcRn binding polypeptide comprises an amino acid sequence as set forth in SEQ ID NO:3 and the second FcRn binding polypeptide comprises an amino acid sequence as set forth in SEQ ID NO:5;
(i) the first FcRn binding polypeptide comprises an amino acid sequence as set forth in SEQ ID NO:4 and the second FcRn binding polypeptide comprises an amino acid sequence as set forth in SEQ ID NO:2;
(j) the first FcRn binding polypeptide comprises an amino acid sequence as set forth in SEQ ID NO:4 and the second FcRn binding polypeptide comprises an amino acid sequence as set forth in SEQ ID NO:3;
(k) the first FcRn binding polypeptide comprises an amino acid sequence as set forth in SEQ ID NO:4 and the second FcRn binding polypeptide comprises an amino acid sequence as set forth in SEQ ID NO:4;
(I) the first FcRn binding polypeptide comprises an amino acid sequence as set forth in SEQ ID NO:4 and the second FcRn binding polypeptide comprises an amino acid sequence as set forth in SEQ ID NO:5;
(m) the first FcRn binding polypeptide comprises an amino acid sequence as set forth in SEQ ID NO:5 and the second FcRn binding polypeptide comprises an amino acid sequence as set forth in SEQ ID NO:2;
(n) the first FcRn binding polypeptide comprises an amino acid sequence as set forth in SEQ ID NO:5 and the second FcRn binding polypeptide comprises an amino acid sequence as set forth in SEQ ID NO:3;
(o) the first FcRn binding polypeptide comprises an amino acid sequence as set forth in SEQ ID NO:5 and the second FcRn binding polypeptide comprises an amino acid sequence as set forth in SEQ ID NO:4; or
(p) the first FcRn binding polypeptide comprises an amino acid sequence as set forth in SEQ ID NO:5 and the second FcRn binding polypeptide comprises an amino acid sequence as set forth in SEQ ID NO:5.

8. The isolated polypeptide of claim 4, wherein the antibody light chain variable region (V L) domain or the antibody heavy chain variable region (V H) domain, or both are human or humanized.

9. A single chain fragment variable (scFv) antibody comprising the isolated polypeptide of claim 1.

10. A vector comprising a polynucleotide encoding the amino acid sequence set forth in SEQ ID NO:1 (QRFX₁TGHFGGLX₂PX₃NG), or (SEQ ID NO:8) YVPKEFNAETFTFH.

11. A polynucleotide encoding the isolated polypeptide of claim 4.

12. A pharmaceutical composition comprising the isolated polypeptide of claim 4 and an excipient.

13. A pharmaceutical composition comprising a population of recombinant bacteria comprising the isolated polypeptide of claim 4.

14. A method of treatment comprising administering a therapeutic antibody comprising the isolated polypeptide of claim 1 to a subject in need thereof.

15. A method of treatment comprising delivering the pharmaceutical composition of claim 12 or 13 to a subject in need thereof.

16. An isolated polypeptide comprising an amino acid sequence set forth in SEQ ID NO:1 (QRFX₁TGHFGGLX₂PX₃NG), wherein the polypeptide is not naturally occurring.

17. The isolated polypeptide of claim 16, wherein X₁ is C or V.

18. The isolated polypeptide of claim 16, wherein X₂ is Y or H.

19. The isolated polypeptide of claim 16 wherein X₃ is C or A.

20. The isolated polypeptide of claim 16, wherein the isolated polypeptide comprises QRFVTGHFGGLHPANG (SEQ ID NO:5).
